(19) United States
(12) Patent Application Publication
(10) Pub. No.: US 2005/0267143 A1
(43) Pub. Date: Dec. 1, 2005

(54) Pyrroloquinoline quinone drugs for treatment of cardiac injury and methods of use thereof

(76) Inventors: Paul J. Davis, West Sand Lake, NY (US); Joel S. Karliner, San Francisco, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/122,572

(22) Filed: May 5, 2005

Related U.S. Application Data
(63) Continuation-in-part of application No. 10/146,566, filed on May 15, 2002.
(60) Provisional application No. 60/568,353, filed on May 5, 2004.
(60) Provisional application No. 60/617,508, filed on Oct. 8, 2004.

(51) Int. Cl. A61K 31/4745

(52) U.S. Cl. 514/291

(57) ABSTRACT

The invention includes compositions comprising substantially purified pyrroloquinoline quinone, that are useful in methods for the treatment and prevention of cardiac injury caused by hypoxia or ischemia. The invention also includes methods for the treatment and prevention of cardiac injury comprising contacting a composition of the invention with a human patient.

### RELATED APPLICATIONS

This application is a continuation-in-part of U.S. application Ser. No. 10/146,566 filed on May 15, 2002, and claims the priority of U.S. Application No. 60/568,353, filed on May 5, 2004 and U.S. Application No. 60/617,508, filed on Oct. 8, 2004, the entire contents of which are incorporated herein by reference.

### BACKGROUND OF THE INVENTION

The heart is critically dependent on uninterrupted blood flow for the delivery of oxygen and nutrients and the removal of harmful products of metabolism. Ischemia leads to rapid changes in myocardial metabolism and cellular injury, the extent of the injury being dependent upon the severity of ischemia. Continued ischemia leads to total tissue necrosis in a few hours.

Reperfusion, although generally considered beneficial, causes tissue injury by several mechanisms. Clinically, in open heart surgery, heart transplantation, and reversal of heart disease, protection of the myocardium against injury by ischemia-reperfusion is an issue of utmost clinical interest. Further, exacerbation of hypoxic injury after restoration of oxygenation (reoxygenation) by reperfusion is an important mechanism of cellular injury in other types of organ transplantation and in hepatic, intestinal, cerebral, renal, and other ischemic syndromes. Cellular hypoxia and reoxygenation cause ischemia-reperfusion injury in part by generating reactive oxygen species (ROS). Since the first isolation of free PQQ from bacteria in the late 1970s, further work has indicated that PQQ is an essential nutrient for vertebrate animals and perhaps belongs to the B group of vitamins (Paz M A, et al. The biomedical significance of PQQ. Edited by Davidson V L: Principles and Applications of Quinoproteins, 1992 by Marcel Dekker, Inc. P381-393, Kasahara T, and Kato T. Nature 2003;422:832). Free PQQ has been identified in red blood cells, neutrophils, cerebrospinal fluid, synovial fluid, bile (Gallop P M, et al. Connect Tissue Res 1993;29:153-161), and in human milk (Mitchell A E, et al. Analytical Biochemistry 1999;269:317-325). Trace amounts of free PQQ have also been detected in spleen, pancreas, lung, brain, heart, intestine, liver, and testis, plasma and urine of humans, and in small intestine, liver, and testis of the rat (Kumazawa T, et al. Biochim Biophys Acta 1992; 1156:62-66). A PQQ-dependent dehydrogenase enzyme is crucial for the amino acid lysine-degradation pathway in mice. In this reaction PQQ acts as a mammalian redox cofactor (Kasahara, T. and Kato, T., Nature 2003;422:832.). Since PQQ levels in human tissues and body fluids are 5-10 times lower than those found in foods, it is probable that PQQ in human tissues is derived, at least partly, from dietary sources including vegetables and meat (Kumazawa T, et al. Biochem J 1995;307:331-333). When mice are fed a PQQ-deficient diet, they grow slowly, have fragile skin and a reduced immune response, and do not reproduce well. It has been shown that PQQ supplementation can improve reproductive performance, growth, and may modulate indices of neonatal extracellular matrix production and maturation in mice fed chemically defined, but otherwise nutritionally complete diets (Steinberg F, et al. Exp Biol Med (Maywood) 2003;228:160-166, Steinberg F M, et al. J Nutr 1994;124:744-753).

Excessive activation of the N-methyl-D-aspartate (NMDA) subtype of the glutamate receptor is critical in the process of neuronal injury in hypoxia/ischemia, and NMDA antagonists can ameliorate neuronal damage in both in vitro and in vivo models of glutamate-mediated neurotoxicity. The results of a previous study demonstrated that PQQ had a protective effect on brain injury in a rodent model of cerebral hypoxia/ischemia and suggested that PQQ could have potential use in the therapy of stroke (Jensen F E, et al. Neuroscience 1994;62(2):399-406). Although PQQ has been shown to be effective in an animal model of focal cerebral ischemia and epilepsy, the protective mechanism is not well understood (Zhang Y, and Rosenberg Pa. European J Neuroscience 2002;16:1015-1024, Jensen F E, et al.).

Only one report investigated the potential cardioprotective effects of PQQ. This study showed that PQQ protected isolated rabbit heart from re-oxygenation injury measured by LDH activity released into the cardiac effluent (Xu F, et al. Biochemical Biophysical Research Communications 1993; 193:434-439). However, based on this information it could not be determined whether PQQ is an effective agent in reducing infarct size when given either prophylactically (pretreatment) or after the onset of ischemia at the time of reperfusion (treatment).

Herein, we are the first to demonstrate that either pretreament or treatment with PQQ can significantly reduce myocardial infarct size in an intact rat model of ischemia or ischemia-reperfusion injury.

### SUMMARY OF THE INVENTION

The present invention relates to the discovery that myocardial oxidative stress can be prevented or minimized by administration of certain cardioprotective factors, and thus has benefit for treating cardiovascular and other diseases. In particular, it has been found that non-toxic dosages of pyrroloquinoline quinone (“PQQ”) drugs are useful as cardioprotective agents, and are therefore valuable in the treatment of a variety of various heart-related ailments such as ischemia-reperfusion injury, congestive heart failure, cardiac arrest and myocardial infarction such as due to coronary artery blockage, and for cardioprotection. PQQ in particular has been found to modulate myocardial oxidative stress such that myocardial cells (which are the subject of the oxidative stress) are protected from cell death.

The compositions and methods of the invention are surprisingly useful for the reduction or elimination of hypoxic/ischemic cardiac injury in vivo and ex vivo, as well as the prevention and/or treatment of cardiovascular disease in mammals in need thereof, such as humans.

In another aspect of the invention, PQQ has been found to modulate, e.g., enhance or maintain the effect of, cardioprotective signaling pathways such as the regulation of the mitochondrial channel mitoK_{ATP}, the nitric oxide-protein kinase C pathway, and the angiotensin-converting enzyme pathway.

In another aspect of the invention, the present invention related to treating or preventing myocardial oxidative stress in myocardial cells in a subject by administering an agent that modulates myocardial oxidative stress such that the myocardial cells are protected from cell death.

In another aspect of the invention, the present invention related to treating or preventing myocardial hypoxic or ischemic damage in a subject by administering an agent that modulates myocardial hypoxic or ischemic damage such that myocardial cells are protected from cell death.

In another aspect of the invention, PQQ has been found to modulate free radical damage caused by myocardial oxidative stress. Free radicals generated by ischemic or hypoxic conditions have been found to be a significant cause of myocardial damage leading to myocardial death. As such, administration of PQQ, administered in vivo in non-toxic dosages, is an effective treatment for inhibiting or preventing myocardial oxidative stress free radical damage.

The invention further relates to methods of improving coronary blood flow in a subject by administering to the subject PQQ in a non-toxic amount, such that coronary blood flow is improved.

In one aspect, the present invention relates to treating or preventing cardiac injury caused by hypoxia or ischemia in a subject by administering pyrroloquinoline quinone, e.g., in an amount effective to treat or prevent cardiac injury. PQQ is typically administered at a non-toxic concentration, e.g., between about 1 nM and less than 10 μM, including less than 900 μM, less than 700 μM, less than 500 μM, less than 300 μM, less than 100 M, or less than 50 μM. In other embodiments, PQQ may be administered at a concentration of about 1 to 10 μM. In other embodiments, PQQ is administered as a function of the subject's body weight. PQQ may typically be administered at a concentration of between about 1 μg/kg to 1 g/kg of a subject's body weight, including less than 500 mg/kg, 250 mg/kg, 100 mg/kg, 10 mg/kg, 5 mg/kg, 3 mg/kg, 2 mg/kg, 1 mg/kg, 500 μg/kg, 250 μg/kg, 100 μg/kg, 10 μg/kg, 5 μg/kg, 2 μg/kg or 1 μg/kg.

The invention further includes cardioprotective agents containing PQQ, e.g., in an amount effective to effect cardioprotection, and a pharmaceutically acceptable carrier. Also included are kits for treating patients at risk of cardiac injury, stroke, or migraine headaches, containing in one or more containers, an effective amount of pyrroloquinoline quinone, a pharmaceutically acceptable carrier, and instructions for use.

In another aspect, the invention relates to treatment or prevention of cardiac injury caused by hypoxia or ischemia in vivo, by administration of an NADPH-dependent methemoglobin reductase substrate; and kits for use in treatment or prevention of cardiac injury, including an effective amount of an NADPH-dependent methemoglobin reductase substrate, a pharmaceutically acceptable carrier, and instructions for use. In some embodiments of the invention the NADPH-dependent methemoglobin reductase substrate is purified from erythrocytes, such as mammalian erythrocytes (e.g., human, bovine, or murine) or non-mammalian erythrocytes (e.g., Rana catesbeiana).

In yet another aspect, the invention relates to methods for preventing organ damage during organ or tissue transplantation, wherein PQQ is administered to an organ donor prior to and/or concurrent with removal of the organ or tissue; and kits for use in preventing organ damage during organ or tissue transplantation, including an effective amount of pyrroloquinoline quinone, a pharmaceutically acceptable carrier, and instructions for use.

In a further aspect, the invention relates to methods for preventing stroke, e.g., in subjects suffering from heart failure, by administering PQQ in amounts effective to obtain the desired protective effect. The PQQ may be desirably administered, e.g., at concentrations of about of about 1 to 10 μM.

The invention includes methods for treating heart failure in a subject by administering PQQ and one or more additional therapeutic compounds. In some embodiments, the additional therapeutic compound may be an anti-platelet drug, anti-coagulant drug and/or an anti-thrombotic drug, or combinations thereof.

In another aspect, the invention relates to methods of treating myocardial infarction in a subject by administering PQQ at levels such that the myocardial infarcfion is decreased or stabilized.

In yet another aspect, the invention relates to methods of preventing migraine headaches in a subject by treating the subject with PQQ. The PQQ may be desirably administered, e.g., at concentrations from about 1 to about 10 μM.

In yet another aspect, the invention relates to methods of preventing reperfusion injury in a subject suffering from or at risk of hypothermia, by treating the subject with PQQ. The PQQ may be desirably administered, e.g., at concentrations from about 1 to about 10 μM.

The invention further relates to methods for preventing vascular occlusion following balloon angioplasty in a subject by pre-treating the subject with PQQ. The subject may be also pre-treated with PQQ and one or more additional therapeutic compounds (e.g., coumadin, angiotensin converting enzyme (ACE) inhibitors such as captopril, benazepril, enalapril, fosinopril, lisinopril, quinapril, ramipril, imidapril, peridopril erbumine and trandolapril, and ACE receptor blockers such as losartan, irbesartan, candesartan cilexetil and valsartan). In some embodiments, the additional therapeutic compound may be an anti-platelet drug, anti-coagulant drug and/or an anti-thrombotic drug, or combinations thereof.

In another aspect, the present invention involves a method for preventing or reducing reperfusion injury in a subject suffering from hypothermic injury by administering PQQ to the subject.

The invention further relates to a pharmaceutical composition for treating myocardial infarction in a subject in need thereof, including a therapeutically effective dose of pyrroloquinoline quinone and a therapeutically effective dose of metoprolol. In one embodiment of the pharmaceutical composition for treating myocardial infarction, the therapeutically effective dose of pyrroloquinoline quinone is 3 mg/kg. In another embodiment of the pharmaceutical composition for treating myocardial infarction, the therapeutically effective dose of metoprolol is 1 mg/kg.

The invention further relates to a kit for treating or preventing a hypoxia or ischemic-related cardiac injury, comprising in one or more containers pyrroloquinoline quinine, metoprolol, a pharmaceutically acceptable carrier, and instructions for use of said kit.

The invention further relates to a method of treating or preventing myocardial oxidative stress in a subject, comprising administering to a subject in need thereof a therapeutically effective dose of pyrroloquinoline quinone and a therapeutically effective dose of metoprolol.

The invention further relates to a method of treating or preventing myocardial infarction in a subject, comprising administering to a subject in need thereof a therapeutically effective dose of pyrroloquinoline quinone and a therapeutically effective dose of metoprolol.

The invention further relates to a method of treating or preventing cardiac injury caused by hypoxia or ischemia in a subject, comprising administering to a subject in need thereof a therapeutically effective dose of pyrroloquinoline quinone and a therapeutically effective dose of metoprolol.

These and other objects of the present invention will be apparent from the detailed description of the invention provided below.

### BRIEF DESCRIPTION OF THE DRAWINGS

FIG. 1 is a bar graph demonstrating the increase in viable adult cardiac mouse myocytes following hypoxia by pretreatment with PQQ.

FIG. 2 is a bar graph showing that PQQ protection is not inhibited by 10 μM 5-hydroxydecanoic acid, a mitochondrial KATP channel inhibitor.

FIG. 3 is a line graph demonstrating that PQQ treatment prior to ischemia preserves left ventricular developed pressure (LVDP).

FIG. 4 is a line graph demonstrating that PQQ treatment prior to ischemia preserves left ventricular end-diastolic pressure (LVEDP); left ventricular systolic pressure minus left ventricular end-diastolic pressure).

FIG. 5 is a line graph demonstrating the effect of PQQ treatment prior to ischemia as measured by the maximum positive first derivative of left ventricular pressure (LVDP).

FIG. 6 is a line graph demonstrating the effect of PQQ treatment prior to ischemia as measured by the maximum negative first derivative of left ventricular pressure (LVDP).

FIG. 7 is a line graph showing that coronary blood flow is significantly improved by PQQ treatment as compared to control.

FIG. 8 is a bar graph indicating that 2 minutes of pretreatment with PQQ at several concentrations shown has progressively favorable responses between 10 nM and 1 μM, but that toxicity occurs at 10 μM.

FIG. 9 is a bar graph demonstrating the changes in cardiac infarction size after PQQ pre-treatment. There is a progressive reduction in infarction size between 10 nM and 1 μM, but infarction size is not reduced at 10 μM PQQ.

FIG. 10 is a schematic showing experimental protocols Model 1 (ischemia 2 hours) and Model 2 (ischemia/reperfusion). Model 2 included two separate sets of experiments; Set 1: (ischemia 17 min/reperfusion 2 hours) and Set 2: (ischemia 30 min/reperfusion 2 hours). The Pretreatment rats received PQQ by i.p. injection before 30 min of ischemia. In the Treatment group PQQ was given by i.v. injection at the onset of reperfusion. Control rats were given an equivalent volume of vehicle at the times indicated. Arrows indicate timing of PQQ administration. i.p.=intraperitoneal; i.v.=intravenous; I=ischemia; LAD=left anterior descending coronary artery.

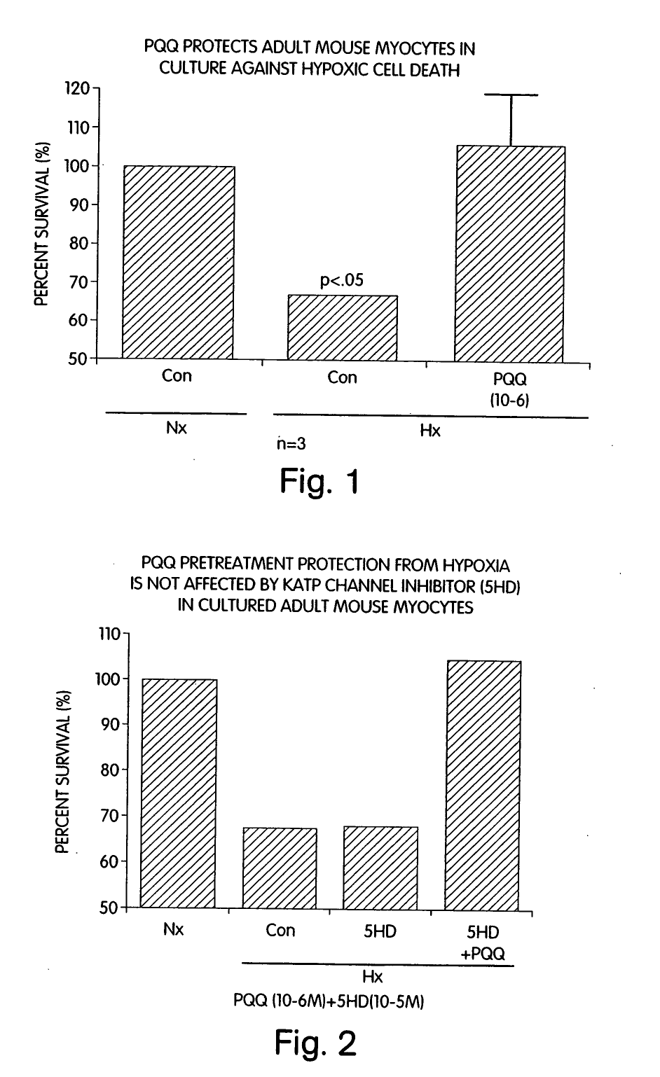

FIG. 1I A is a line graph showing left ventricular systolic pressure (LVSP) in model 2 rats during ischemia/reperfusion. Either pretreatment with PQQ (PQQ by i.p. injection before 30 min of ischemia) or treatment with PQQ (PQQ by i.v. injection at the onset of reperfusion) resulted in increased LVSP at 2 hours of reperfusion. B=baseline; I=ischemia; R=reperfusion.

FIG. 11B is a line graph showing left ventricular developed pressure (LVDP) in model 2 rats during ischemia/reperfusion. Treatment with PQQ increased LVDP after both 30 min and 2 hours of reperfusion. Pretreatment with PQQ increased LVDP at 2 hours of reperfusion. B=baseline; I=ischemia; R=reperfusion.

FIG. 12A is a line graph showing left ventricular (LV) (+)dP/dt in model 2 rats during ischemia/reperfusion. Either pretreatment with PQQ or treatment with PQQ significantly increased LV (+)dP/dt at 2 hours of reperfusion. B=baseline; I=ischemia; R=reperfusion.

FIG. 12B is a line graph showing left ventricular (LV) (−)dP/dt in model 2 rats during ischemia/reperfusion. Either pretreatment with PQQ or treatment with PQQ significantly decreased LV (−)dP/dt at 2 hours of reperfusion. B=baseline; I=ischemia; R=reperfusion.

FIG. 13 is a bar graph showing myocardial infarct size in model 1 (ischemia only). Pretreatment with PQQ 20 mg/kg significantly reduced infarct size (Infarct mass/LV mass %). Ischemia was induced by 2 hours of LAD ligation without reperfusion.

FIG. 14 is a bar graph showing myocardial infarct size in model 2 (ischemia/reperfusion). In these experiments ischemia was induced by 17 min of LAD occlusion followed by 2 hours of reflow (reperfusion). Pretreament with PQQ 20 mg/kg significantly reduced infarct size (as measured either by Infarct mass/Risk area % or Infarct mass/LV mass %).

FIG. 15 is a bar graph showing myocardial infarct size in additional experiments in Model 2 (ischemia/reperfusion). In these experiments 30 min of ischemia was followed by 2 hours of reperfusion. Either pretreatment with PQQ 15 mg/kg or treatment with PQQ 15 mg/kg significantly reduced infarct size (as measured either by Infarct mass/Risk area % or Infarct mass/LV mass %). P values refer to respective I/R infarct size measurements.

FIG. 16 is a line graph showing effects of pretreatment with different doses of PQQ on infarct size in five groups of rats pretreated with the indicated range of PQQ doses by i.p. injection. There was a strong negative relationship between infarct size and the dose of PQQ.

FIG. 17A is a bar graph showing average episodes of ventricular fibrillation (VF) per rat in combining data from both model 1 and model 2. Pretreatment with PQQ 15-20 mg/kg significantly decreased average episodes of VF per rat. Analysis was by one-way analysis of variance (ANOVA).

FIG. 17B is a bar graph showing the percentage of rats with VF using combined data from model 1 and model 2. Either pretreatment with PQQ 15-20 mg/kg or treatment with PQQ 15-20 mg/kg significantly decreased the percentage of rats with VF. Analysis was by the Fisher Exact test.

FIG. 18A is a line graph showing myocardial MDA levels from the anterior segment of the LV subjected to 30 min of LAD occlusion followed by 2 hours of reperfusion. Pretreatment with PQQ 15 mg/kg significantly decreased MDA in the ischemic myocardium. Differences between rats subjected to I/R and treated or not (Control) were significant by two-way analysis of variance. Sham=rats subjected to LAD coronary artery isolation without occlusion for the total study period.

FIG. 18B is a line graph showing myocardial MDA levels from the posterior (non-ischemic) segment of the LV. Pretreatment with PQQ 15 mg/kg also decreased MDA in this non-ischemic remote myocardium.

FIG. 19 is a bar graph showing respiratory control ratios of mitochondria isolated from rat hearts under the following conditions: (i) Controls: 3 hours pentobarbital anesthesia, n=4, (ii) PQQ treatment: 3 mg/kg, 20 min equilibration period, 30 min ischemia, PQQ injection, 2 hrs reperfusion, n=5; and (iii) ischemia/reperfusion: 20 min equilibration period, 30 min ischemia followed by 2 hrs reperfusion, n=3.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

“Ischemia” includes the decrease or cessation of myocardial blood flow.

“Hypoxia” includes the deficiency in the amount of oxygen reaching body tissues.

“Hypoxia or ischemic-related injury” includes cardiac injury.

“Reperfusion” includes the restoration of blood flow to an organ or tissue that has had its blood supply cut off, as after a heart attack or stroke.

“Oxidative stress” includes conditions that occur when there is an excess of free radicals, a decrease in antioxidant levels, or both.

“Necrosis” includes the death of cells or tissues through injury or disease, particularly in a localized area of the body such as the myocardium.

“Apoptosis” refers to programmed cell death.

“Beta blockers” include agents such as atenolol, metoprolol, and propranolol, which act as competitive antagonists at the adrenergic beta receptors. Such agents also include those more selective for the cardiac (beta-1) receptors which allows for decreased systemic side effects. Beta blockers reduce the symptoms connected with hypertension, cardiac arrhythmias, migraine headaches, and other disorders related to the sympathetic nervous system. Beta blockers also are sometimes given after heart attacks to stabilize the heartbeat. Within the sympathetic nervous system, beta-adrenergic receptors are located mainly in the heart, lungs, kidneys, and blood vessels. Beta blockers compete with the nerve-stimulating hormone epinephrine for these receptor sites and thus interfere with the action of epinephrine, lowering blood pressure and heart rate, stopping arrhythmias, and preventing migraine headaches.

“Cardiac injury” includes any chronic or acute pathological event involving the heart and/or associated tissue (e.g., the pericardium, aorta and other associated blood vessels), including ischemia-reperfusion injury; congestive heart failure; cardiac arrest; myocardial infarction; cardiotoxicity caused by compounds such as drugs (e.g., doxorubicin, herceptin, thioridazine and cisapride); cardiac damage due to parasitic infection (bacteria, fungi, rickettsiae, and viruses, e.g., syphilis, chronic Trypanosoma cruzi infection); fulminant cardiac amyloidosis; heart surgery; heart transplantation; and traumatic cardiac injury (e.g., penetrating or blunt cardiac injury, aortic valve rupture).

“Subject” includes living organisms such as humans, monkeys, cows, sheep, horses, pigs, cattle, goats, dogs, cats, mice, rats, cultured cells therefrom, and transgenic species thereof. In a preferred embodiment, the subject is a human. Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing cardiac injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of pyrroloquinoline quinone which allow it to perform its intended function, e.g., treating or preventing, partially or totally, cardiac injury caused by hypoxia or ischemia as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Specific binding” or “specifically binds” includes proteins, such as an antibody which recognizes and binds an pyrroloquinoline quinone or a ligand thereof, but does not substantially recognize or bind other molecules in a sample.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Pharmaceutically acceptable esters” includes relatively non-toxic, esterified products of therapeutic compounds of the invention. These esters can be prepared in situ during the final isolation and purification of the therapeutic compounds or by separately reacting the purified therapeutic compound in its free acid form or hydroxyl with a suitable esterifying agent; either of which are methods known to those skilled in the art. Acids can be converted into esters according to methods well known to one of ordinary skill in the art, e.g., via treatment with an alcohol in the presence of a catalyst.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

“Unit dose” includes a discrete amount of the pharmaceutical composition comprising a predetermined amount of the active ingredient.

Pyrroloquinoline quinone (PQQ) is a water soluble anionic quinone that can transfer electrons catalytically between a variety of reductants and oxidants, and may be part of a soluble electron transport system in eukaryotic cells. PQQ proper is of the general structure

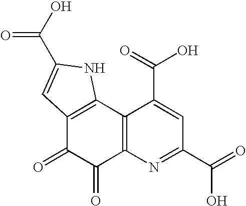

“Pyrroloquinoline quinone” includes any member of the pyrroloquinoline quinone family having chemical similarity, including closely related isomeric and stereoisomeric analogs of PQQ (See e.g., Zhang et al., 1995, Biochem. Biophys. Res. Commun. 212: 41-47, 1995). PQQ is also known as methoxatin. PQQ is found in animal tissues and fluids. Without wishing to be bound by theory, PQQ may act in part as a free-radical scavenger, particularly of reactive oxygen species (ROS). As such, PQQ may function as an NADPH-dependent methemogloblin reductase substrate (See e:g., Xu et al., Proc. Natl. Acad. Sci. USA, 1992, 89(6):2130-4). Other NADPH-dependent methemogloblin reductase substrates may function to decrease or eliminate hypoxia or ischemia-related cardiac injury.

Compositions comprising substantially purified pyrroloquinoline quinone may include pyrroloquinoline quinone alone, or in combination with other components such as beta blockers, and compounds which are effective to favorably modulate cardioprotective signaling pathways such as phenylephrine, sphingosine-1-phosphate, or the ganglioside GM-1. Pyrroloquinoline quinone may be substantially purified by any of the methods well known to those skilled in the art. (See, e.g., E. J. Corey and Alfonso Tramontano, J. Am. Chem. Soc., 103, 5599-5600 (1981); J. A. Duine, Review Ann. Rev. Biochem. 58, 403 (1989)).

The pyrroloquinoline quinone of the invention is, in one embodiment, a component of a pharmaceutical composition, which may also comprise buffers, salts, other proteins, and other ingredients acceptable as a pharmaceutical composition. The invention also includes a modified form of pyrroloquinoline quinone, which is capable of preventing or reducing hypoxic/ischemic cardiac injury as described herein.

The structure of the therapeutic compounds of this invention may include asymmetric carbon atoms. It is to be understood accordingly that the isomers (e.g., enantiomers and diastereomers) arising from such asymmetry are included within the scope of this invention. Such isomers can be obtained in substantially pure form by classical separation techniques and by sterically controlled synthesis. For the purposes of this application, unless expressly noted to the contrary, a therapeutic compound shall be construed to include both the R or S stereoisomers at each chiral center. In certain embodiments, a therapeutic compound of the invention comprises a cation. If the cationic group is hydrogen, H⁺, then the therapeutic compound is considered an acid. If hydrogen is replaced by a metal ion or its equivalent, the therapeutic compound is a salt of the acid. Pharmaceutically acceptable salts of the therapeutic compound are within the scope of the invention, e.g., pharmaceutically acceptable alkali metal (e.g., Li⁺, Na⁺, or K⁺) salts, ammonium cation salts, alkaline earth cation salts (e.g., Ca²⁺, Ba²⁺, Mg²⁺), higher valency cation salts, or polycationic counter ion salts (e.g., a polyammonium cation). (See, e.g., Berge et al. (1977) “Pharmaceutical Salts”, J. Pharm. Sci. 66:1-19). It will be appreciated that the stoichiometry of an anionic compound to a salt-forming counter ion (if any) will vary depending on the charge of the anionic portion of the compound (if any) and the charge of the counter ion. Preferred pharmaceutically acceptable salts include a sodium, potassium or calcium salt, but other salts are also contemplated within their pharmaceutically acceptable range.

The invention relates to methods of treating or preventing myocardial oxidative stress, such as is caused by hypoxia or ischemia, in a subject. This is done by administering to a subject in need thereof a preferably non-toxic amount of an agent such as PQQ which modulates myocardial oxidative stress such that the myocardial cells which are the target of the oxidative stress are protected from cell death. The cell death may be due, e.g., to necrosis or apoptosis.

Cardioprotective signaling pathways are known in the art. These pathways may be targeted for enhancement in patients in need of cardioprotection, by administering, pyrroloquinoline quinone in an amount effective to enhance or maintain the effect of cardioprotective signaling pathway.

Free radicals generated by ischemic or hypoxic conditions have been found to be a significant cause of myocardial damage leading to myocardial death. As such, administration of PQQ, administered in vivo, e.g., in non-toxic dosages, is an effective treatment for inhibiting or preventing myocardial oxidative stress free radical damage, either by PQQ-mediated free radical scavenging, or by inhibition of free radical generation.

Administration of the compounds of the invention may be done where clinically necessary or desirable, e.g., at the onset of reperfusion, or prior to reperfusion.

It has surprisingly also been found that coronary flow may be beneficially improved in a subject, e.g., one suffering from a low blood flow condition, by administering to a subject in need thereof a non-toxic amount of pyrroloquinoline quinone. This is illustrated in the Examples. Coronary flow may be measured by several indicators, such as the left ventricular diastolic pressure (“LVDP”) or the left ventricular (“LVEDP”). Measurement of coronary flow, such as by determining LVDP or LVEDP, is within the skill of those in the art.

Cardiac injury caused by hypoxia or ischemia, such as myocardial infarction, may therefore be treated or prevented by administration of pyrroloquinoline quinone, preferably in a non-toxic dosage, e.g., at a concentration of less than about 10 μM.

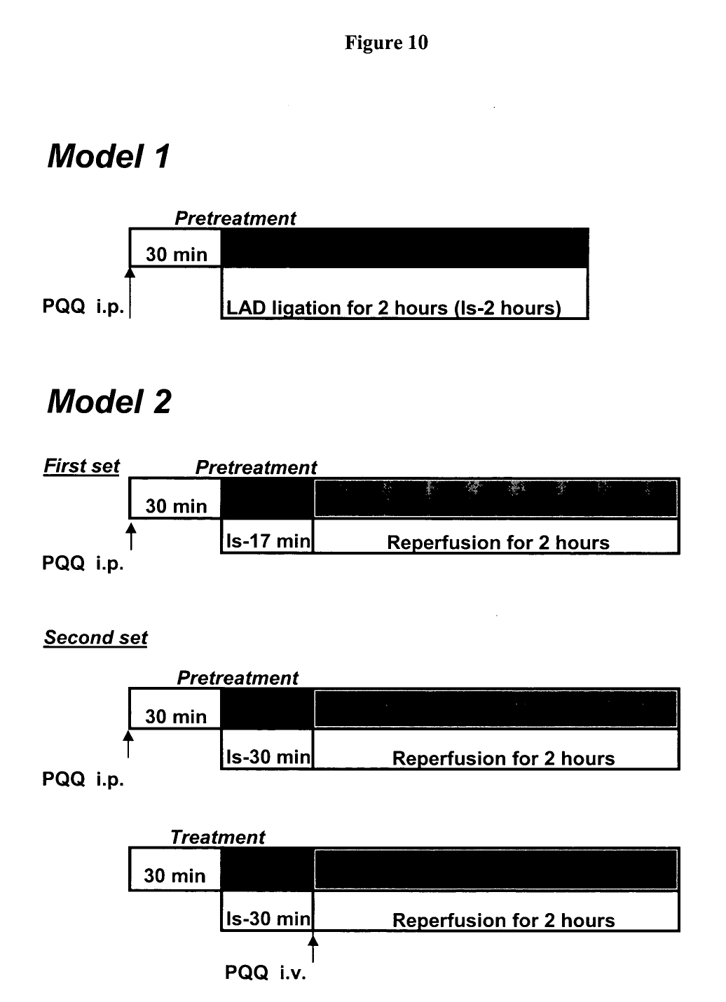

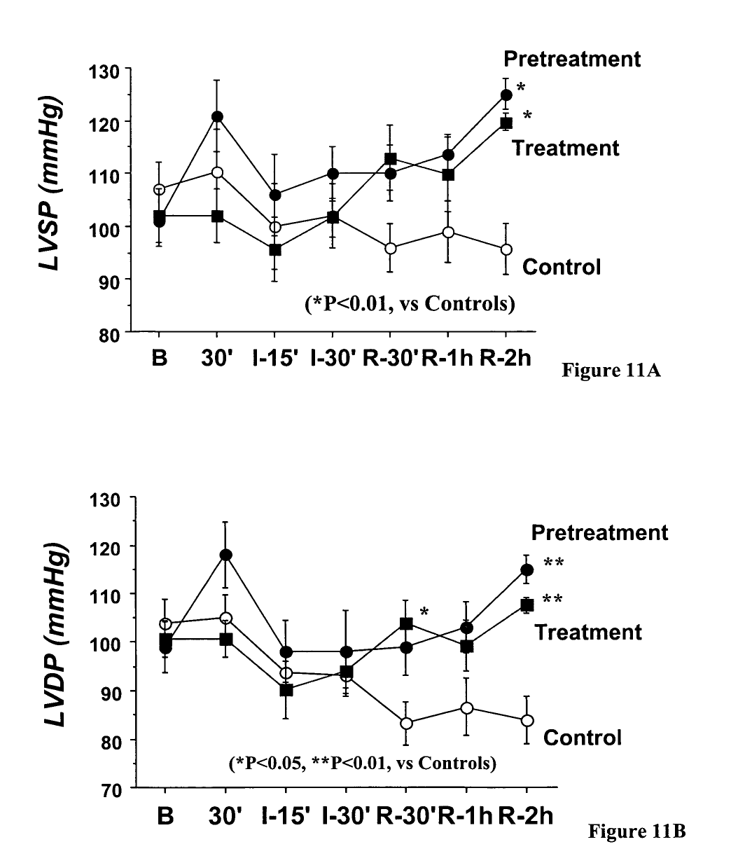

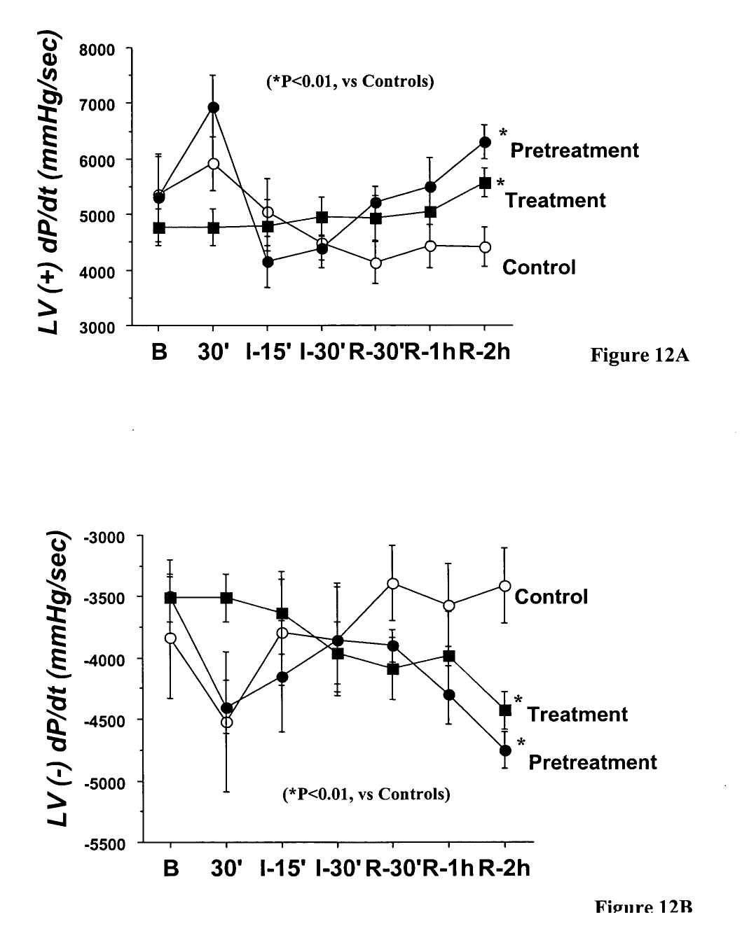

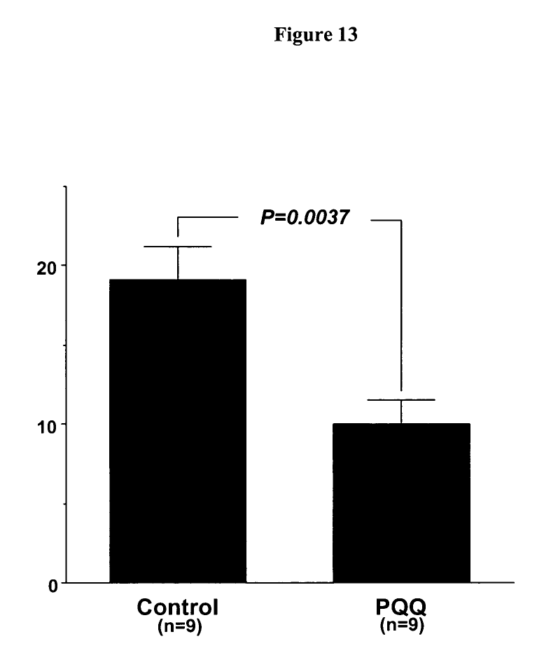

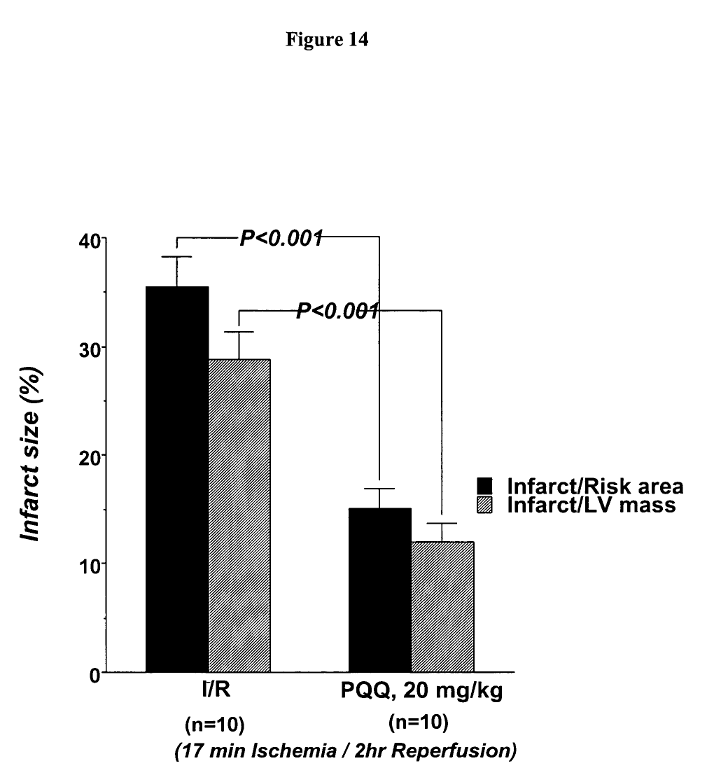

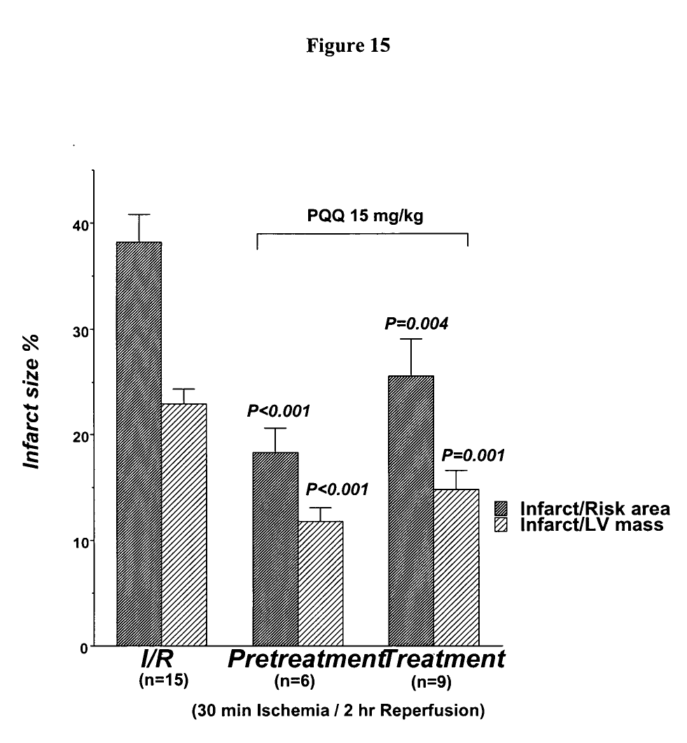

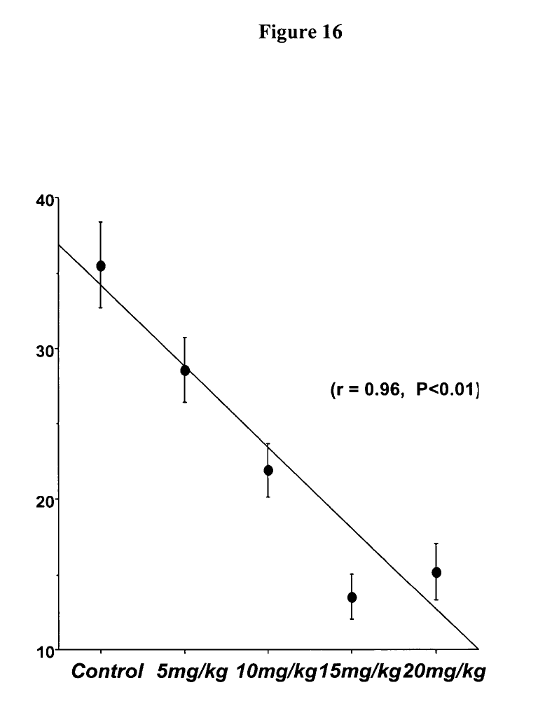

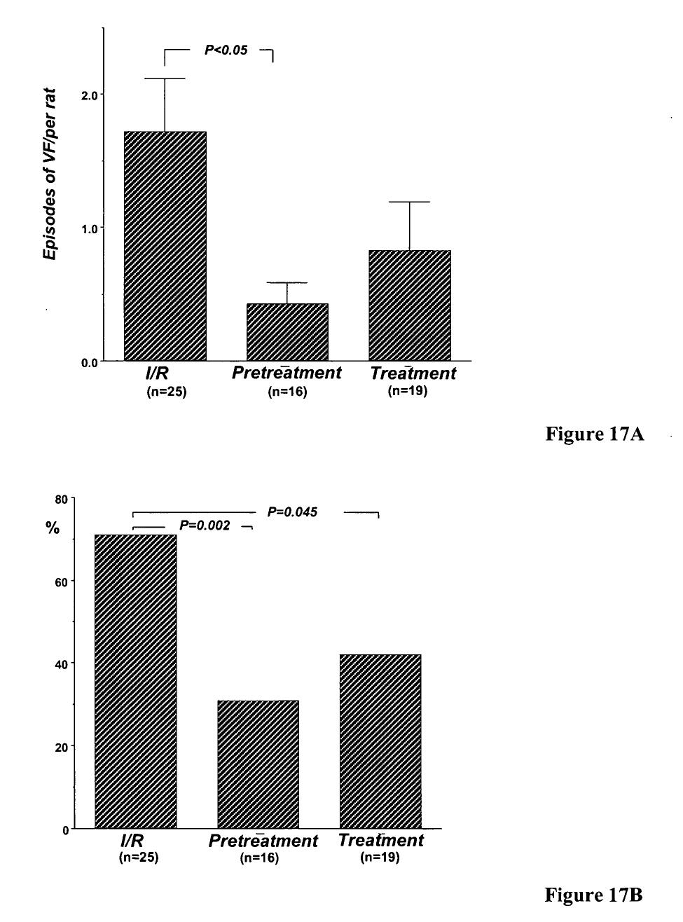

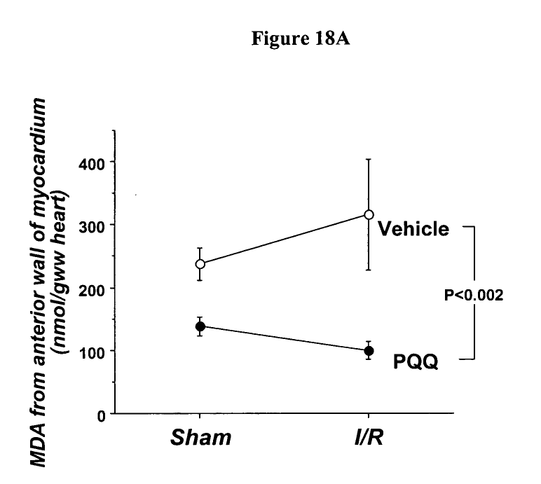

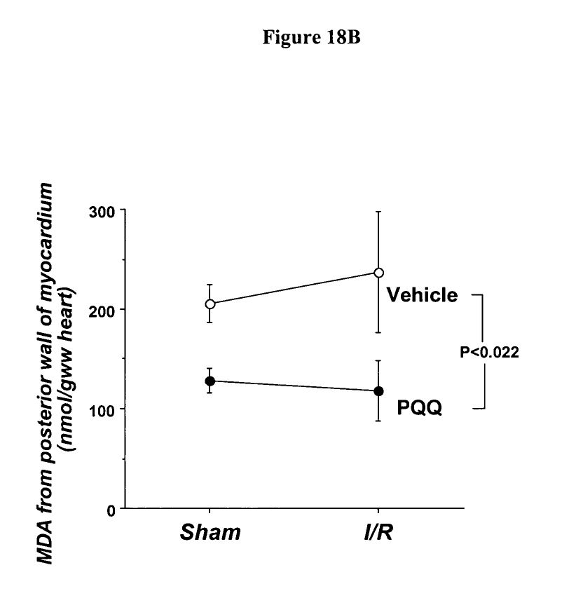

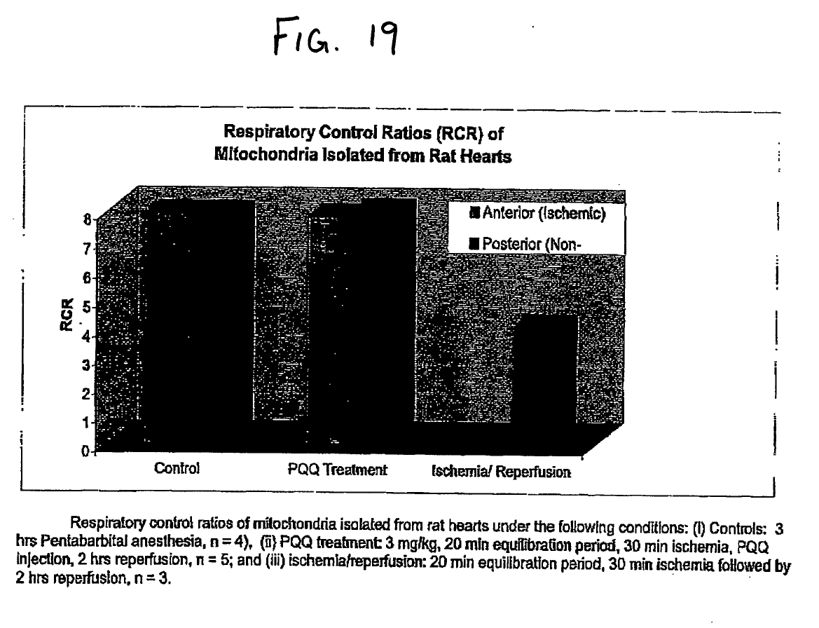

Rats were subject to PQQ treatment subject to two different models. In model 1 (shown schematically in FIG. 10A), male Sprague-Dawley rats were subjected to 2 hours of left anterior descending (LAD) coronary artery ligation without reperfusion. In model 2 (ischemia-reperfusion, shown in FIG. 10B), rats were subjected to 17 or 30 minutes of LAD occlusion and 2 hours of reperfusion with left ventricular (LV) hemodynamic monitoring. PQQ (15-20 mg/kg) was given either 30 min before LAD occlusion by i.p. injection (Pretreatment) or by i.v. injection at the onset of reperfusion (Treatment) to mimic the clinical state in humans. Controls received vehicle (2% NaHCO₃).

In model 1, infarct size (infarct mass/LV mass) after PQQ treatment was smaller than control (PQQ treatment resulted in infarct size of 10.0±1.5 vs control of 19.1±2.1%, n=9, P<0.01). In model 2, either pretreatment or treatment with PQQ resulted in reduced infarct size (infarct mass/risk area) (PQQ pretreatment infarct size 18.4±2.3 and treatment infarct size 25.6±3.5% vs control of 38.1±2.6%, P<0.01). PQQ protected against ischemia-induced cardiac dysfunction with higher LV developed pressure, LV (+)dP/dt and lower LV (−)dP/dt after 1-2.

In summary, PQQ had cardioprotective effects in two separate intact rat infarction models consisting either of ischemia or ischemia-reperfusion. PQQ reduced infarct size either when given prior to ischemia or ischemia-reperfusion, or when given at the onset of reperfusion. Moreover, PQQ had beneficial hemodynamic effects as evidenced by increased left ventricle (LV) developed pressure and LV (+)dP/dt at 1-2 hours of reperfusion. Pretreatment with PQQ decreased average episodes of ventricular fibrillation (VF) per rat and the percentage of rats with VF while treatment with PQQ decreased the percentage of rats with VF during ischemia and reperfusion. The dose of PQQ was inversely related to infarct size. PQQ reduced levels of malondialdehyde (MDA), an index of lipid peroxidation, in ischemic myocardium.

Mortality during the ischemia-reperfusion period in the three groups in model 2 tended to decrease after PQQ (Control: 28.6%, Pretreatment: 12.9%, Treatment: 21.7%). However, these results did not reach statistical significance. It should be noted that the study focused on measurements of infarct size and hemodynamics and was not designed as a mortality trial which would have required a much larger number of animals.

PQQ is also effective when given at the onset of reperfusion. Studies of myocardial tissue levels of malondialdehyde (MDA), a lipid peroxidation product that reacts with thiobarbituric acid have been completed. Ischemia/reperfusion augmented MDA levels and PQQ prevented this increase. The values comparing the PQQ and sham controls after ischemia/reperfusion (I/R) differed by 3-fold. Similar effect was seen in the remote “normal” myocardium.

PQQ given either as pretreatment or as treatment at the onset of reperfusion is highly effective in reducing myocardial infarct size and improving cardiac function in a dose-related manner in rat models of ischemia and ischemia-reperfusion. The malondialdehyde (MDA) results showing that this indicator of lipid peroxidation was reduced by PQQ, suggest that PQQ acts as a free radical scavenger in ischemic myocardium.

While not wishing to be bound by theory, one possible mechanism of PQQ action is that PQQ acts as a free radical scavenger. Recent studies indicate that PQQ functions as a free radical scavenger in addition to acting as a cofactor of quinoprotein enzymes (Urakami T, et al. J Nutr Sci Vitaminol (Tokyo) 1997;43:19-33, He K, et al. Biochemical Pharmacology 2003;65:67-74). PQQ can act as a neuroprotectant by suppressing peroxynitrite formation (Zhang Y and Rosenberg Pa.). PQQ was an effective antioxidant protecting mitochondria against oxidative stress-induced lipid peroxidation, protein carbonyl formation and inactivation of the mitochondrial respiratory chain (He K, et al., Miyauchi K, et al. Antioxid Redox Signal 1999;1:547-554.). Phagocytic cells, such as monocytes and neutrophils, generate superoxide in response to stimuli. Several inhibitors of redox cycling of PQQ were demonstrated to be blocking agents for superoxide release by both stimulated neutrophils and monocytes. This suggests that PQQ is involved in the respiratory burst of both macrophages and neutrophils (Bishop A, et al. Free Radic Bio Med 1995;18:617-620, Bishop A, et al. Free Radic Bio Med 1994; 17:311-320).

Our results are consistent with the above studies. We found that pretreatment with PQQ significantly decreased myocardial MDA levels in the infarct zone and in the remote “normal” myocardium. Our data indicate that MDA was increased by I/R in this putative normal area and are in accord with reports by others indicating the presence of LV dysfunction in remote myocardium during acute ischemia in humans and animals (Yang Z, et al. Circulation 2004;109:1161-1167, Kramer C M, et al. Circulation 1996; 94:660-666.). Our observations are consistent with the hypothesis that PQQ reduces lipid peroxidation and inactivates superoxide in both ischemic and non-ischemic myocardium. Therefore, the protective effect of PQQ on ischemia-reperfusion injury can be due to its action as a free radical scavenger. In our study PQQ given either as pretreatment or treatment also reduced the incidence of ventricular fibrillation. Furthermore, PQQ may have a direct antiarrhythmic effect, and may cause a reduction in VF due to its anti-ischemic properties.

The data, shown in Example 5 below, suggest that PQQ reduces lipid peroxidation as well as scavenges superoxide. Further evidence that the protective effect of PQQ on I/R injury is due to its action as a free radical scavenger. The observation that MDA increased after I/R in the putative normal area is consistent with observations reported by others that shows both in humans and in animal models that myocardium remote from the infarct zone exhibits depressed function.

The least dose of PQQ that is effective in reducing infarct size at the time of reperfusion was also determined as shown in Example 5. In addition we have determined the effect of this dose of PQQ on mitochondrial function. For these studies a previously described method of tissue preparation was used. The heart was visually divided longitudinally into an anterior portion comprising the territory perfused by the left anterior descending coronary artery and non-infarcted posterior portion. Rats were treated or not (sham controls) with PQQ after 30 min of ischemia immediately before the onset of 2 hr of reperfusion (I/R).

Treatment at the onset of reperfusion with 1 mg/kg PQQ did not protect either the heart or isolated intact mitochondria from I/R injury. However, treatment with only 3 mg/kg PQQ was highly effective in reducing infarct size by 49% and in restoring mitochondrial respiration. In these experiments hemodynamic results did not differ from those described in previous reports.

Our data also indicate that either prophylactic administration of PQQ in high-risk patients or treatment at the time of an active ischemic episode is of benefit by reducing infarct size and ventricular arrhythmias. Our results also indicate that treatment with PQQ is also effective at the time of reperfusion as occurs with chemical thrombolysis or balloon angioplasty/stenting when these procedures are employed as early treatment of acute myocardial infarction. The absence of depressant effects on systemic hemodynamics in this study is also encouraging. Further exploration in other models of ischemia-reperfusion injury, where free radical generation is a paramount cause of damage may be desired. Acute toxicity, especially adverse effects on renal function that have been described in rats (Watanabe A, et al. Hiroshima J Med Sci 1989; 38:49-51), and the potential benefit of PQQ in humans, if any, remains to be determined.

Thus, PQQ given either as pretreatment before ischemia or as treatment at the onset of reperfusion following ischemia is highly effective in reducing myocardial infarct size and improving cardiac function in a dose-related manner in intact rats. PQQ appears to act as a free radical scavenger in ischemic myocardium.

Metoprolol is a β₁-selective (cardioselective) adronoceptor blocking agent. It reduces oxygen demand of the heart, slowing the heart rate and reducing cardiac output at rest and upon exercise; reduces systolic blood pressure, among other things. The drug is available in the United States as the tartrate salt (LOPRESSOR™, Geigy Pharmaceuticals), as 50 mg and 100 mg tablets. The effective daily dose is 100 mg to 450 mg, and LOPRESSOR™ is usually dosed as 100 mg given in two daily doses. Metoprolol is also available as 50 mg, 100 mg and 200 mg extended release tablets in the United States as the succinate salt (TOPROL XL™, Astra Pharmaceutical Products, Inc.), which may be dosed once daily.

PQQ may be coadministered with metoprolol. Results shown in Example 6, below, show that the combined use of PQQ and metoprolol tended to reduce infarct size greater than PQQ or metoprolol alone. In one embodiment, metoprolol is administered in a 1:3 ratio with the dose of PQQ. For example, a 3 mg/kg dose of PQQ is accompanied by a 1 mg/kg dose of metoprolol. In another embodiment, the metoprolol is administered at a daily dose from about 50 mg to about 450 mg combined with a daily dose of PQQ from about 50 mg to about 500 mg per day. Myocardial oxidative stress can be prevented or minimized by administration of a combination of PQQ and metoprolol, and thus has benefit for treating cardiovascular and other diseases. In particular, combinations of PQQ and metoprolol are useful as cardioprotective agents, and are therefore valuable in the treatment of a variety of various heart-related ailments such as ischemia-reperfusion injury, congestive heart failure, cardiac arrest and myocardial infarction such as due to coronary artery blockage, and for cardioprotection. The combinations in particular are useful for modulating myocardial oxidative stress such that myocardial cells (which are the subject of the oxidative stress) are protected from cell death.

The invention encompasses methods of treating or preventing cardiac injury caused by hypoxia or ischemia in a subject, wherein PQQ is administered to a subject in need thereof, such that hypoxia or ischemic-related injury is prevented or decreased. In certain embodiments, the PQQ is administered at a concentration of less than about 10 μM. In other embodiments, the PQQ is administered at a concentration in the range of about 10 nM to about 10 μM, about 10 nM to about 1 μM, 100 nM to about 10 μM, and 100 nM to about 500 nM. In still other embodiments of the invention, the PQQ is administered at a concentration such that the concentration of PQQ at the site of cardiac tissue is in the range of 10 nM to about 10 μM. PQQ may also be administered as a function of the subject's body weight. In some embodiments of the invention, PQQ is administered at a concentration of between about 1 μg/kg to 1 g/kg of a subject's body weight, including less than 500 mg/kg, 250 mg/kg, 100 mg/kg, 10 mg/kg, 5 mg/kg, 3 mg/kg, 2 mg/kg, 1 mg/kg, 500 μg/kg, 250 μg/kg, 100 μg/kg, 10 μg/kg, 5 μg/kg, 2 μg/kg or 1 μg/kg. In further embodiments of the invention, the PQQ is administered at a non-toxic concentration, which includes concentrations of PQQ which are cytostatic but not cytotoxic, and concentrations which are cytotoxic to cell types other than the intended one or more cell types (e.g., cardiomyocytes). The determination of the cytotoxicity of a known concentration of PQQ to one or more cell types is within the abilities of one of ordinary skill in the art. By way of non-limiting example, toxicity to cultured adult mouse cardiac myocytes is observed at a concentration of 100 μM PQQ. In some embodiments, PQQ is administered in combination with other compounds, such as anti-platelet drugs, anti-coagulant drugs, and anti-thrombotic drugs.

The cardiac injury that can be treated or prevented by the methods and compositions of the present invention includes all cardiac injury caused or affected by hypoxia and/or ischemia. Such injury includes, but is not limited to, ischemia-reperfusion injury, congestive heart failure, myocardial infarction, cardiotoxicity caused by compounds such as drugs (e.g., doxorubicin), cardiac damage due to parasitic infection, fulminant cardiac amyloidosis, heart surgery, heart transplantation, and traumatic cardiac injury. All or a portion of the heart may be injured, including associated blood vessels and/or tissue, such as the pericardium.

The invention also encompasses a method of treating or preventing cardiac injury caused by hypoxia or ischemia in a subject, by administering to a subject in need thereof an NADPH-dependent methemoglobin reductase substrate, such that said hypoxia or ischemic-related injury is prevented or decreased. In embodiments of the invention, the NADPH-dependent methemoglobin reductase substrate is purified from erythrocytes, such as mammalian erythrocytes (e.g., human, bovine, or murine) or non-mammalian erythrocytes (e.g., Rana catesbeiana). One of ordinary skill in the art will know how to isolate and purify NADPH-dependent methemoglobin reductase substrates with minimal experimentation.

The invention further encompasses a method of preventing organ or tissue damage during organ or tissue transplantation, by administering to a donor pyrroloquinoline quinone prior to or concurrent with removal of said organ or tissue, such that damage caused by reperfusion of said organ or tissue is decreased or prevented. In preferred embodiments, the organ or tissue to be transplanted is the heart or cardiac tissue. The PQQ may also be contacted with the organ or tissue following surgical removal of the organ or tissue from the donor. In some embodiments, the PQQ is added in addition to known organ or tissue preservation solutions, such as University of Wisconsin solution or Celsior solution (See, e.g., Thabut et al., Am J Respir Crit Care Med, 2001, 164(7):1204-8; Faenza et al., Transplantation, 2001,72(7):1274-7).

The invention still further encompasses methods of preventing stroke in a subject (e.g., a human) suffering from heart failure, by treating a subject with pyrroloquinoline quinone and a pharmaceutically acceptable carrier. In some embodiments, the pyrroloquinoline quinone is administered to the subject at a concentration of less than about 10 μM. The PQQ may be administered prior to, or concomitant with, a surgical procedure that may increase the likelihood of a stroke in the patient. In one embodiment, the procedure is balloon angioplasty. Other procedures include coronary artery bypass surgery and valve replacement surgery. The PQQ may be administered prior to, concomitant with, or after anti-thrombogenic agents (e.g., coumadin).

The invention also encompasses methods of reducing or preventing headaches in a subject (such as a human), by treating the subject with pyrroloquinoline quinone and a pharmaceutically acceptable carrier. Such headaches include acute and chronic migraine headaches and sinus headaches.

The invention still further encompasses a method of preventing reperfusion injury in a subject (such as a human) suffering from hypothermia, by treating the subject with pyrroloquinoline quinone and a pharmaceutically acceptable carrier. The subject may be treated with PQQ prior to or concomitant with the standard rewarming procedures for treating a person suffering from hypothermia as are generally known in the art.

As noted above, combination therapies of PQQ and metoprolol are part of the invention. The combination therapies of the invention are administered in any suitable fashion to obtain the desired treatment of myocardial infarction in the patient. Substantially simultaneous administration can be accomplished, for example, by administering to the subject a single infusion having a fixed ratio of a PQQ and, metoprolol, or in multiple, single injections. The components of the combination therapies, as noted above, can be administered by the same route or by different routes. For example, a PQQ is administered orally, while the metoprolol is administered intravenously; or all therapeutic agents may be administered by intravenous injection. The sequence in which the therapeutic agents are administered is not believed to be critical.

Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Thus, the compounds of the invention and the other pharmacologically active agent may be administered to a patient simultaneously, sequentially or in combination. If administered sequentially, the time between administrations generally varies from 0.1 to about 48 hours. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

The beneficial effect of the combination composition of the invention includes, but is not limited to, pharmacokinetic or pharmacodynamic co-action resulting from the combination of therapeutic agents. In one embodiment, the co-action of the therapeutic agents is additive. In another embodiment, the co-action of the therapeutic agents is synergistic. In another embodiment, the co-action of the therapeutic agents improves the therapeutic regimen of one or both of the agents.

The invention further relates to kits for treating patients suffering a myocardial infarction, comprising a therapeutically effective dose of at least one metoprolol, and a PQQ, either in the same or separate packaging, and instructions for its use.

To evaluate whether a patient is benefiting from the (treatment), one would examine the patient's symptoms in a quantitative way, by decrease in the frequency of relapses, or increase in the time to sustained progression. In a successful treatment, the patient status will have improved, measurement number or frequency of relapses will have decreased, or the time to sustained progression will have increased.

As for every drug, the dosage is an important part of the success of the treatment and the health of the patient. In every case, in the specified range, the physician has to determine the best dosage for a given patient, according to gender, age, weight, height, pathological state and other parameters.

The pharmaceutical compositions of the present invention contain a therapeutically effective amount of the active agents. The amount of the compound will depend on the patient being treated. The patient's weight, severity of illness, manner of administration and judgment of the prescribing physician should be taken into account in deciding the proper amount. The determination of a therapeutically effective amount of an PQQ or metoprolol is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

The invention encompasses the preparation and use of pharmaceutical compositions comprising a compound useful for the prevention or reduction of hypoxic/ischemic cardiac injury as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a pharmaceutically acceptable ester or salt, such as in combination with a physiologically-acceptable cation or anion, as is well known in the art. Further, the pyrroloquinoline quinone may contain pharmacologically acceptable additives (e.g., carrier, excipient and diluent), stabilizers or components necessary for formulating preparations, which are generally used for pharmaceutical products, as long as it does not adversely affect the efficacy of the preparation, e.g., in decreasing or inhibiting ischemia or reperfusion injury.

Examples of additives and stabilizers include saccharides such as monosaccharides (e.g., glucose and fructose), disaccharides (e.g., sucrose, lactose and maltose) and sugar alcohols (e.g., mannitol and sorbitol); organic acids such as citric acid, maleic acid and tartaric acid and salts thereof (e.g., sodium salt, potassium salt and calcium salt); amino acids such as glycine, aspartic acid and glutamic acid and salts thereof (e.g., sodium, calcium or potassium salt); surfactants such as polyethylene glycol, polyoxyethylene-polyoxypropylene copolymer and polyoxyethylenesorbitan fatty acid ester; heparin; and albumin.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. The preferred mode is intravenous administration.

The pyrroloquinoline quinone and the above-mentioned ingredients are admixed as appropriate to give powder, granule, tablet, capsule, syrup, injection and the like. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. The amount of the active ingredient is generally equal to the dosage of the active ingredient, which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Particularly contemplated additional agents include anti-emetics and scavengers such as cyanide and cyanate scavengers. Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include potato starch and sodium starch glycollate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in, e.g., U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, a gel or cream or solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

Additional delivery methods for administration of compounds include a drug delivery device, such as that described in U.S. Pat. No. 5,928,195.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally-administrable formulations that are useful include those, which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations that are useful include those, which comprise the active ingredient in microcrystalline form or in a liposomal preparation.

The mixture of pyrroloquinoline quinone and pharmacologically acceptable additives is preferably prepared as a lyophilized product, and dissolved when in use. Such preparation can be prepared into a solution containing about 0.01-100.0 mg/ml of pyrroloquinoline quinone, by dissolving same in distilled water for injection or sterile purified water. More preferably, it is adjusted to have a physiologically isotonic salt concentration and a physiologically desirable pH value (pH 6-8).

While the dose is appropriately determined depending on symptom, body weight, sex, animal species and the like, it is generally assumed that treatment options holding the blood concentration at about 1 μM will be preferred. This plasma concentration may be achieved through administration of one to several doses a day. When pyrroloquinoline quinone is to be administered to a subject, 0.1 ng to 10 mg/kg body weight (e.g., 1 ng to 1 mg/kg body weight) of pyrroloquinoline quinone can be given intravenously.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

These Examples are provided for the purpose of illustration only and the invention should in no way be construed as being limited to these Examples, but rather should be construed to encompass any and all variations which become evident as a result of the teaching provided herein.

### Example 1

### In Vitro Studies of PQQ Preservation of Cardiac Myocyte Viability

An in vitro model of cultured adult cardiac mouse myocytes was developed to study cardioprotection by PQQ. These cells are viable in culture for up to 48 hours at a physiologic pH and consist of >90% rod-shaped cells. These cells can be used readily for determination of cell viability by trypan blue exclusion, and for biochemical, immunochemical, and molecular studies. In this model, approximately 35% of the cells die when exposed to 0% oxygen in a hypoxia chamber for 2-3 hours. As shown in FIG. 1, 1 μM PQQ added 1 hour before subjecting the cells to severe hypoxia (0% oxygen for 2-3 hours) produces a significant increase in the proportion of viable cells as indicated by trypan blue exclusion. A higher concentration of PQQ (100 μM) is highly toxic under normoxic conditions as evidenced by 100% cell death. FIG. 2 demonstrates that 1 μM PQQ protection against hypoxia-induced cell death is not inhibited by 10 μM 5-hydroxydecanoic acid, a mitrochondrial KATP channel inhibitor. Without wishing to be bound by theory, these data suggest that PQQ does not exert cardioprotection by opening mitochondrial KATP channels.

### Example 2

### Ex Vivo Studies of PQQ Preservation of Cardiac Function

Ex vivo studies were performed using an isolated mouse heart preparation employing the Langendorff technique. In this approach, the heart is removed and mounted on a perfusion apparatus in which drugs can be given via an aortic cannula. The heart is paced at a constant rate, and left ventricular developed pressure [LVDP; left ventricular systolic pressure minus left ventricular end-diastolic pressure], left ventricular end-diastolic pressure [LVEDP], and the maximum positive and negative first derivatives of left ventricular pressure [+dP/dtmax and −dP/dtmax] are recorded. The heart is equilibrated for 20 min. After drug or vehicle is infused, the heart is subjected to 20 min of ischemia [coronary flow completely stopped] followed by 30 min of reperfusion. Coronary sinus flow as a reflection of coronary blood flow is also measured. This protocol leads to severe myocardial injury as measured by hemodynamic parameters.

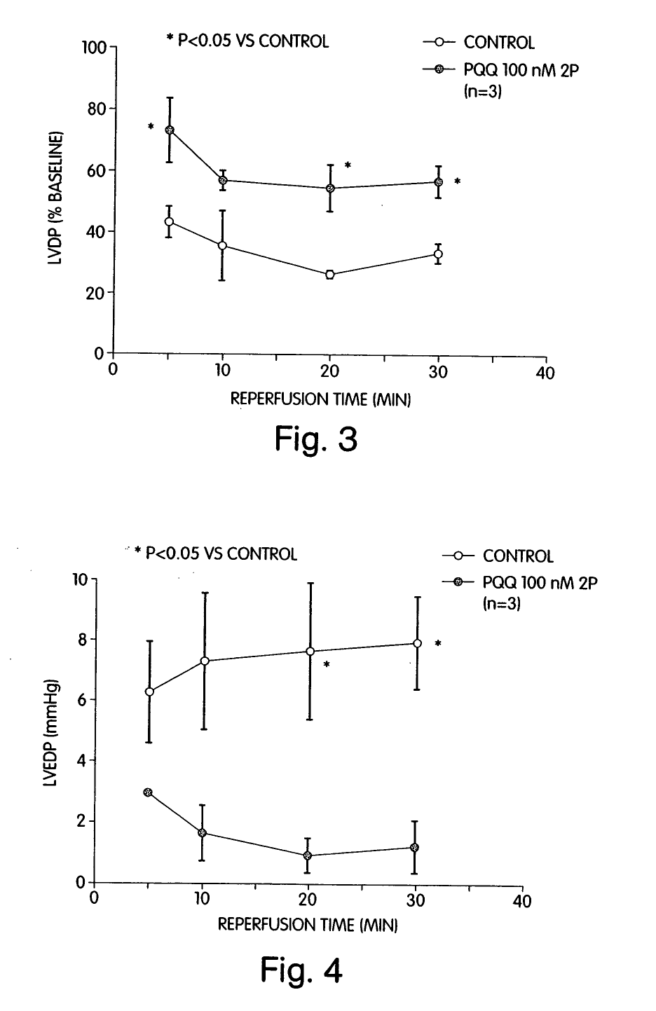

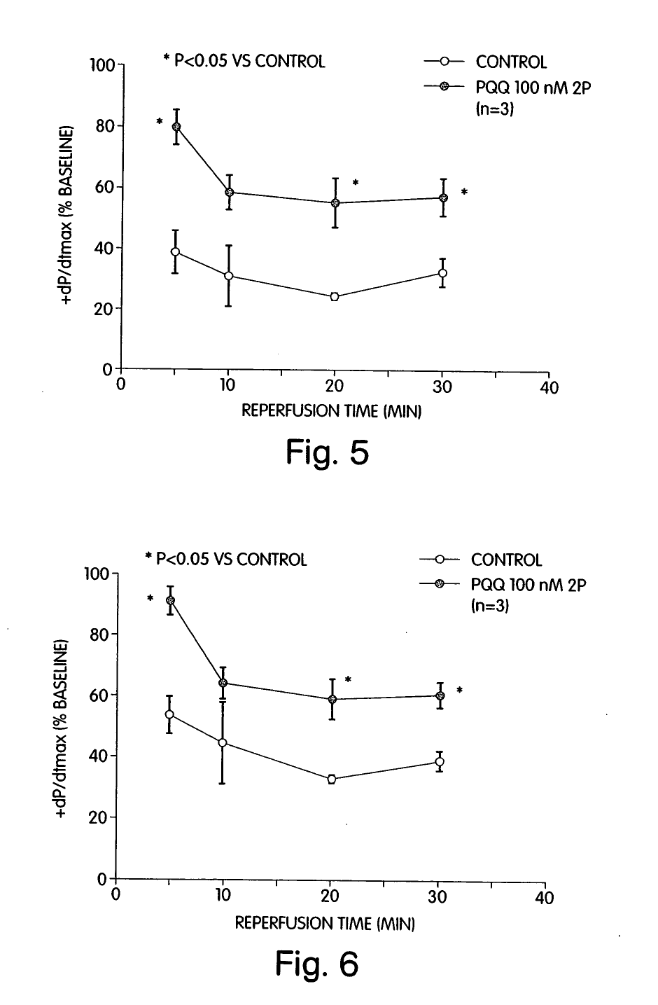

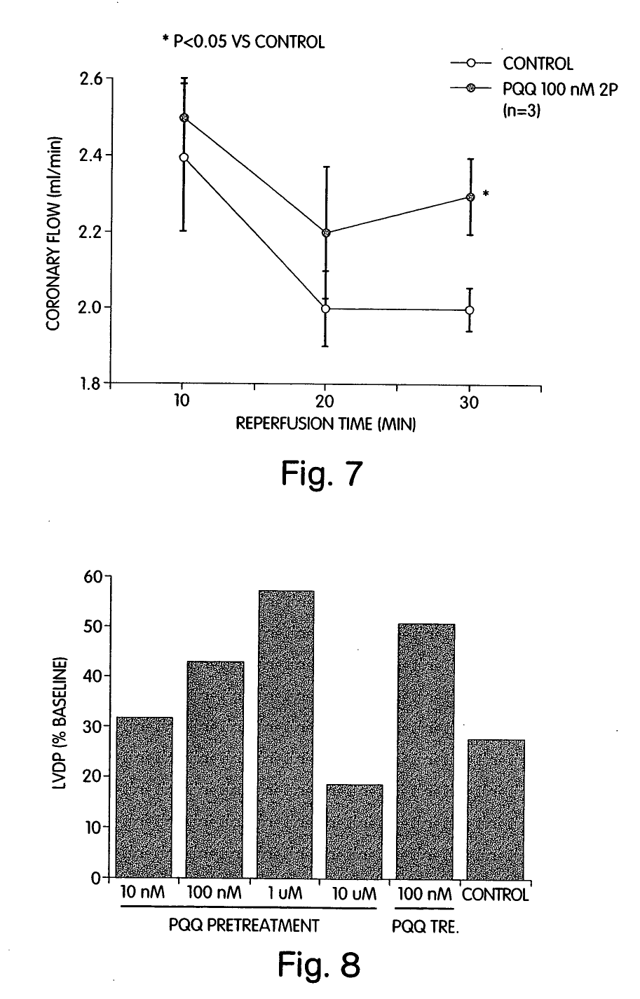

As seen in FIG. 3, 100 nM PQQ infused for only 2 minutes prior to complete cessation of coronary blood flow produces significant preservation of LVDP Baseline 1. VDP averages 60 mmHg. Similar results are obtained with LVEDP [FIG. 4] Baseline LVEDP averages 8 mm Hg. (Note that an increase in LVEDP represents an adverse response). As expected, the data for + and −dP/dtmax track the LVDP results [FIGS. 5 and 6]. Similarly, coronary flow is significantly improved by PQQ pretreatment compared to control [FIG. 7].

In FIG. 8, it is shown that 2 min of pretreatment with PQQ at the concentrations shown has progressively favorable responses between 10 nM and 1 μM, but that toxicity occurs at 10 μM. Of major interest is that 100 nM PQQ given at the time of onset of reperfusion (PQQ tre.) is equivalent to pretreatment. Therefore, PQQ is useful in both pretreatment, e.g., in cardiac or other surgical procedures, and after symptoms occur, e.g., in the acute critical cardiac events.

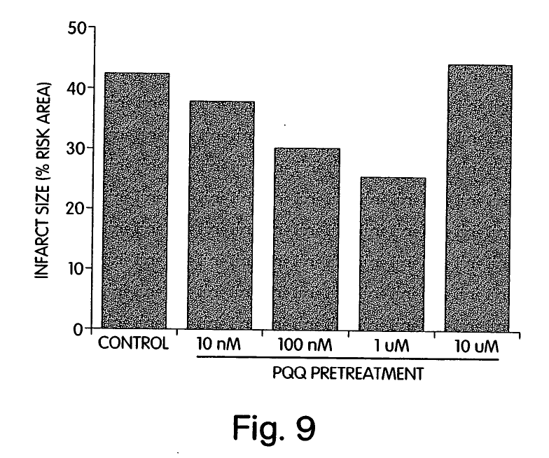

FIG. 9 shows the results of experiments of infarct size measurements after PQQ pretreatment. As indicated, there is a progressive reduction in infarct size between 10 nM and 1 μM, paralleling the hemodynamic data. Consistent with the latter, infarct size is not reduced at 10 μM PQQ.

### Example 3

### PQQ Preservation of Oxidatively Stressed Cells

Cultured cardiac myocytes are subjected to oxidative stress by in vitro administration of H₂O₂. Two studies are done, one in which PQQ is added in concentrations between 10 nM and less than 10 μM to cardiac myocytes, after which H₂O₂ is added. In the other study, cardiac myocytes are subjected to insult in vitro administration of H₂O₂ for two hours, after which PQQ is added in concentrations between 10 nM and less than 10 μM. In both studies, PQQ is found to be protective.

### Example 4

### Use of PQQ for Prevention/Reduction of Oxidative Stress in Vivo

Male Sprague-Dawley rats were randomly treated with pyrroloquinoline quinone (PQQ) either before ischemia or ischemia-reperfusion. PQQ (15-20 mg/kg) was given 30 min before left anterior descending coronary artery (LAD) occlusion by intraperitoneal injection (pretreatment) or at the onset of reperfusion by intravenous injection (treatment). Rats were subjected to 17 or 30 min of LAD occlusion and 2 hours of reperfusion with left ventricle (LV) hemodynamic monitoring. PQQ given either as pretreatment or treatment decreased infarct size in these rat models. PQQ protected against ischemia-induced cardiac dysfunction with higher LV developed pressure, LV (+)dP/dt and lower LV (−)dP/dt after 1-2 hour of reperfusion. There were fewer episodes of ventricular fibrillation (VF) in PQQ treated rats. Myocardial malondialdehyde (MDA), an indicator of lipid peroxidation, was reduced by PQQ. Thus, PQQ given either as pretreatment or as treatment at the onset of reperfusion is highly effective in reducing myocardial infarct size and improving cardiac function in a dose-related manner in rat models of ischemia and ischemia-reperfusion. The MDA results suggest that PQQ acts as a free radical scavenger in ischemic myocardium.

Statistical Analysis.

All results are presented as mean±SEM. The two treatment groups (pretreatment and treatment) were compared with the normal control group using one-way analysis of variance (ANOVA) with the regression equation for multiple group comparisons. Differences in mortality during the occlusion and reperfusion period among the three groups were assessed by the Chi-square test. The percentages of rats with VF were assessed by the Fisher Exact test. All computations were done using the general linear model procedure in Minitab, version 7.2 (Minitab Statistical Software) or Primer of Biostatistics: The program, version 3.03 (McGraw-Hill). Statistical significance was set at p<0.05.

Models of Ischemia and Ischemia-Reperfusion.

PQQ was dissolved in vehicle (2% NaHCO₃). The volume given either intraperitoneally (i.p.) or intravenously (i.v.) was one ml. All controls were treated with one ml of vehicle. In model 1, PQQ at 20 mg/kg was given i.p. 30 min before 2 hours of ischemia induced by LAD ligation. In model 2, PQQ at 15 mg/kg was given i.p. 30 min before either 17 or 30 min of ischemia followed by 2 hours of reperfusion (pretreatment). In other model 2 experiments, PQQ at 15 mg/kg was given at the onset of reperfusion by i.v. bolus injection via the femoral vein (treatment). These protocols are summarized in FIG. 10.

After induction of anesthesia (ketamine 80 mg/kg, xylazine 4 mg/kg body weight intraperitoneally), a tracheostomy was performed and the animal was ventilated on a Harvard Rodent Respirator (Model 683, Harvard Apparatus). Model 1 rats were subjected to 2 hours of proximal left anterior descending (LAD) coronary artery ligation without reperfusion. Model 2 employed ischemia-reperfusion as previously described (Sievers R E, et al. Magn Reson Med 1989; 10:172-81). In this model, a reversible coronary artery snare occluder was placed around the proximal LAD coronary artery through a midline sternotomy. Rats were then subjected to 17 or 30 minutes of LAD occlusion and 120 minutes of reflow. In addition, model 2 rats had hemodynamic measurements recorded. A 4F Millar catheter was inserted through the right carotid artery into the left ventricle (LV). After 20 min of equilibration, heart rate (HR), systolic pressure (LVSP), end diastolic pressure (LVEDP), LV (+)dP/dt max, and LV (−)dP/dt max were monitored using a MacLab/4S (Milford, M A). LV developed pressure (LVDP) was calculated by subtracting LVEDP from LVSP.

Body weights among the three groups of rats in both model 1 and model 2, sets 1 and 2 did not differ (values for Control, Pretreatment, and Treatment groups were: 320±16, 321±22, and 306±18 gm, respectively; p=0.799 by analysis of variance (ANOVA)).

There were no significant differences in heart rate, LVSP, LVEDP, LV (+)dP/dt, and LV (−)dP/dt among control, pretreatment and treatment groups in model 2 at baseline. Whether given as pretreatment or treatment, PQQ protected against ischemia-induced cardiac dysfunction with higher LVSP, LVDP, LV (+)dP/dt and lower LV (−) dP/dt after 1-2 hours of reperfusion (FIGS. 11A-B; 12A-B).

Infarct Size.

Infarct size was measured as described previously (Sievers R E, et al. Magn Reson Med 1989; 10:172-81, Zhu B-Q, et al. J Am Coll Cardiol 1997; 30:1878-85). In model 1, hearts were excised at the end of the 2 hour ischemic period. The sections were then incubated in a 1% solution of triphenyltetrazolium chloride (TTC) for 10 to 15 min until viable myocardium was stained brick red.

In model 2, after 2 hours of reperfusion, the LAD was reoccluded, and phthalocyanin dye (Engelhard Cooperation, Louiseville, Ky.) was injected into the LV cavity, allowing normally perfused myocardium to stain blue. The heart was then excised, rinsed of excess dye and sliced transversely from apex to base into 2-mm-thick sections. The sections were incubated in TTC as described above. Infarcted myocardium fails to stain with TTC. The tissue sections were then fixed in a 10% formalin solution and weighed. Color digital images of both sides of each transverse slice were obtained using a videocamera (Leica DC 300 F) connected to a microscope (Stereo Zoom 6 Photo, Leica). The regions showing blue-stained (nonischemic), red-stained (ischemic but noninfarcted) and unstained (infarcted) tissue were outlined on each color image and measured using NIH Image 1.59 (National Institutes of Health, Bethesda, Md.) in a blinded fashion. On each side, the fraction of the LV area representing infarct-related tissue (average of two images) was multiplied by the weight of that section to determine the absolute weight of infarct-related tissue. The infarct size for each heart was expressed as:

$$Infarct size / LV mass ( \% ) = \frac{Σ Infarct weight in each slice}{Total LV weight} \times 100 \% , Risk area / LV mass ( \% ) = \frac{\begin{matrix}Total weight of \\ non \text{-} blue \text{-} stained section\end{matrix}}{Total LV weight} \times 100 \% , Infarct size as a percentage of risk area was then calculated as \frac{Σ Infarct weight in each slice}{Σ Risk area weight of each slice} \times 100 \%$$

In the ischemic model (model 1), infarct size (infarct mass/LV mass, without phthalocyanin blue dye injected) after PQQ was smaller than Control (FIG. 13). In the first set of experiments in model 2, ischemia was for 17 min followed by 2 hours of reperfusion, infarct size (infarct mass/risk area, infarct mass/LV mass) was reduced by pretreatment with PQQ 20 mg/kg (FIG. 14). In the second set of model 2 experiments, ischemia was for 30 min followed by 2 hours of reperfusion Infarct size after either Pretreatment or Treatment with PQQ 15 mg/kg was smaller than Control (FIG. 15).

FIG. 16 shows that the dose of PQQ given as Pretreatment was inversely related to infarct size. In these experiments, 17 min of ischemia was followed by 2 hours of reperfusion.

Ventricular Fibrillation (VF).

Electrocardiograms (ECGs, lead II) were obtained by inserting subcutaneous needle electrodes into the limbs. The ECG was monitored during ischemia and reperfusion and episodes of paroxysmal VF were recorded. The number episodes of VF per rat and the percentage of rats with VF in each group were calculated. No rat received antiarrhythmics before or during occlusion and reperfusion. Episodes of VF were successfully treated by rapidly striking the exposed myocardium with the thumb and index finger of one hand.

Pretreatment with PQQ at 15 or 20 mg/kg decreased average episodes of VF per rat (FIG. 17A). Either Pretreatment or Treatment with PQQ at 15 or 20 mg/kg decreased the percentage of rats with VF (FIG. 17B).

In separate additional experiments in which malondialdehyde was measured (see below) there were no episodes of VF in either the Sham or PQQ groups (n=5 each). However, VF in the I/R group (30 min of ischemia followed by 2 hours reperfusion) averaged 1.8±0.4 episodes/rat and was reduced by Pretreatment with PQQ 15 mg/kg to 0.2±0.2 episodes/rat (n=5 each group, P<0.001 by two-way ANOVA with a significant interaction between PQQ and I/R P=0.002).

Myocardial Malondialdehyde (MDA) Measurement.

Myocardial tissue MDA, a lipid peroxidation product that reacts with thiobarbituric acid, was determined spectrophotometrically at an absorbance of 532 nm. The concentration of the samples was calculated using an extinction coefficient of 1.56×105/M cm and the results were expressed as nmol/g wet weight heart (Ohkawa H, et al. Analytical Biochemistry 1979;95:351-358, Moritz F, et al. Cardiovascular Research 2003;59:834-843).

For measurements of myocardial tissue MDA, an indicator of lipid peroxidation, a different method of tissue preparation was used. The heart was divided visually from apex to base into an anterior portion comprising the territory perfused by the LAD and the remaining non-ischemic portion. Rats were pretreated or not (Sham controls) with 15 mg/kg PQQ and subjected to 30 min of ischemia and 2 hours of reperfusion. In these additional experiments hemodynamic results did not differ from those described above. As can be seen in FIG. 18A, I/R augmented MDA levels and PQQ prevented this increase. PQQ and Control values after I/R differed by 3-fold (FIG. 18A). A similar effect was seen in the remote “normal” myocardium (FIG. 18B). When given at the onset of reperfusion, PQQ 15 mg/kg also reduced MDA levels in ischemic myocardium from 176±16 to 123±17 nmol/g (n=8, P<0.05).

### Example 5

### PQQ Restores Mitochondrial Respiration at Low Doses and is Cardioprotective in In Vivo Models of Ischemia/Reprefusion Injury

Adult male rats underwent 30 min of left anterior descending coronary artery (LAD) occlusion and 2 hrs of reperfusion. To assess the potential benefits of reperfusion therapy in humans, PQQ was given by i.v. injection at doses of 1 and 3 mg/kg bodyweight at the onset of reperfusion. After removal, the hearts were divided from apex to base into an anterior part comprising the LAD-perfused territory and a posterior segment, and mitochondria were isolated. Mitochondrial respiration of each myocardial segment was measured and compared to that of mitochondria isolated from preconditioned (without PQQ) and sham operated hearts.

Treatment with 1 mg/kg PQQ did not protect mitochondria from I/R injury. However, treatment with 3 mg/kg PQQ was highly effective in restoring mitochondrial respiration. The respiratory control and ADP-to-oxygen consumption ratios (RCR: 8.0±0.5, ADP/O; 4.5), and state 3 respiration rates (RR: 41 nmol O atom/min/mg protein, n=5) of the ischemic areas matched those of the shams (RCR: 8.2±0.3, ADP/O: 3.7, RR=43 nmol O atom/min/mg protein), while the RCR values of hearts preconditioned without PQQ were surprisingly 20% lower. Respiratory responses from mitochondria of ischemic, untreated hearts were reduced by 50-100%. Electron micrographs of PQQ-treated tissue and mitochondria did not reveal the morphology typical of myocardial damage. PQQ also reduced infarct size and myocardial malondialdehyde (MDA) tissue levels by 49% and 61%, respectively.

Low-dose, 3 mg/kg PQQ given at reperfusion very effectively restores mitochondrial respiration inhibited by ischemia and reduces oxidative damage to mitochondria and infarct size in I/R injury. PQQ may exert its cardioprotective function as a lipid peroxidation inhibitor or radical scavenger. Thus, PQQ treatment may emerge as a powerful therapy of acute ischemic syndromes.

The respiratory control and ADP-to-oxygen consumption ratios (RCR: 8.0 0.5, ADP/O; 4.5), and state 3 respiration rates (RR: 41 nmol O atom/min/mg protein, n=5) of the ischemic areas matched those of the shams, while the RCR values of hearts preconditioned without PQQ were surprisingly 20% lower. Respiratory responses from mitochondria of ischemic, untreated hearts were reduced by 50-100%. RCR data are shown graphically in FIG. 19. Electron micrographs of PQQ-treated tissue and mitochondria did not reveal the morphology typical of myocardial damage. Thus, low-dose 3 mg/kg PQQ given at reperfusion very effectively restores mitochondrial respiration inhibited by ischemia and reduces oxidative damage to mitochondria and infarct size in I/R injury.

Toxicity studies were also carried out. We found no renal or hepatic toxicity at doses of either 3 mg/kg or 10 mg/kg (see Table 1). As noted above, PQQ 3 mg/kg given at the time of reperfusion appears to be an effective cardioprotective dose. At 15 mg/kg, 7/8 animals showed no renal or hepatic toxicity, but one rat excluded from the data presented in Table 1 did develop uremia at 4 days and was dead at 10 days. Rats that received 20 mg/kg had received 10 mg/kg two weeks previously for a cumulative dose of 30 mg/kg. As indicated in Table 1, all of these animals developed uremia and were dead at 10 days. All laboratory studies were performed in a blinded fashion by the clinical laboratory at the San Francisco VA Medical Center. Note that baseline levels of some measures, such as albumin, are lower in rats than in humans, while others, such as creatine kinase (CK), are higher.

TABLE 1

| PQQ | BUN | CRE | NA | K | CL | CO2 | TP |
|---|---|---|---|---|---|---|---|
| 3 mg/kg | (n = 4) | | | | | | |
| Baseline | 16.5 ± 1.0 | 0.57 ± 0.03 | 140 ± 0.9 | 5.2 ± 0.6 | 103 ± 0.4 | 27 ± 1.4 | 5.8 ± 0.1 |
| 4 days | 16.3 ± 1.1 | 0.38 ± 0.03 | 141 ± 0.7 | 5.9 ± 0.3 | 104 ± 0.7 | 25 ± 0.8 | 5.8 ± 0.1 |
| 10 days | 14.3 ± 0.3 | 0.35 ± 0.03 | 139 ± 0.5 | 4.8 ± 0.5 | 104 ± 1.0 | 27 ± 0.3 | 6.0 ± 0.2 |
| 10 mg/kg | (n = 6) | | | | | | |
| Baseline | 16.3 ± 1.3 | 0.38 ± 0.05 | 135 ± 1.9 | 8.2 ± 2 | 101 ± 1.0 | 25 ± 2 | 5.4 ± 0.2 |
| 4 days | 17.3 ± 0.8 | 0.38 ± 0.03 | 135 ± 2.1 | 11.6 ± 2 | 101 ± 0.3 | 24 ± 2 | 6.0 ± 0.1 |
| 10 days | 14.8 ± 0.5 | 0.38 ± 0.03 | 138 ± 1.0 | 5.5 ± 0.7 | 102 ± 0.7 | 27 ± 1 | 6.0 ± 0.1 |
| 15 mg/kg | (n = 7)& | | | | | | |
| Baseline | 17.9 ± 0.5 | 0.34 ± 0.02 | 140.4 ± 0.5 | 4.7 ± 0.2 | 100.8 ± 0.9 | 29.4 ± 0.6 | 5.9 ± 0.1 |
| 4 days | 17.4 ± 1.5 | 0.44 ± 0.05 | 140.6 ± 0.8 | 4.8 ± 0.5 | 101.1 ± 0.7 | 29.6 ± 0.5 | 5.9 ± 0.1 |
| 10 days | 16.0 ± 1.0 | 0.38 ± 0.02 | 140.3 ± 0.7 | 5.5 ± 0.8 | 100.5 ± 1.0 | 30.2 ± 0.5 | 6.08 ± 0.1 |
| 20 mg/kg | (n = 4)@ | | | | | | |
| Baseline | 18.5 ± 1.0 | 0.36 ± 0.03 | 139 ± 0.7 | 5.2 ± 0.4 | 102 ± 0.8 | 28 ± 2 | 6.0 ± 0.1 |
| 4 days | 390 ± 2.6** | 9.7 ± .07** | 129 ± 4.3 | 11.8 ± 0.6** | 88 ± 1.0# | 6 ± 1** | 4.8 ± 0.1* |
| 10 days | Died | | | | | | |

| PQQ | ALB | ALP | AST | ALT | CK | BW(g) |
|---|---|---|---|---|---|---|
| 3 mg/kg | | | | | | |
| Baseline | 1.7 ± 0.04 | 256 ± 6.1 | 82 ± 1.3 | 52 ± 5 | 873 ± 240 | 357.5 ± 7.5 |
| 4 days | 1.5 ± 0.08 | 206 ± 5.5# | 100 ± 19 | 58 ± 2 | 775 ± 258 | 377.5 ± 7.8 |
| 10 days | 1.6 ± 0.03 | 215 ± 15 | 83 ± 10 | 62 ± 3 | 712 ± 325 | 395 ± 8.9# |
| 10 mg/kg | | | | | | |
| Baseline | 1.7 ± 0.03 | 305 ± 24 | 79 ± 21 | 43 ± 1 | 1113 ± 831 | 350 ± 11 |
| 4 days | 1.7 ± 0.07 | 305 ± 29 | 95 ± 22 | 60 ± 2 | 902 ± 441 | 368.7 ± 10 |
| 10 days | 1.6 ± 0.04 | 273 ± 33 | 85 ± 22 | 50 ± 2 | 2240 ± 1450 | 387.6 ± 11 |
| 15 mg/kg | | | | | | |
| Baseline | 1.7 ± 0.07 | 365 ± 48 | 81.4 ± 6.7 | 50 ± 3 | 507 ± 109 | 334 ± 16 |
| 4 days | 1.5 ± 0.08 | 293 ± 46 | 77.3 ± 8.0 | 56 ± 5 | 640 ± 353 | 364 ± 18 |
| 10 days | 1.6 ± 0.1 | 294 ± 39 | 70.0 ± 4.9 | 50 ± 3 | 726 ± 209 | 370 ± 20 |
| 20 mg/kg | | | | | | |
| Baseline | 1.6 ± 0.03 | 211 ± 9.5 | 97 ± 15 | 55 ± 3 | 872 ± 443 | 402.5 ± 7.8 |
| 4 days | 1.1 ± 0.04** | 124 ± 5.8* | 173 ± 16# | 54 ± 2 | 1331 ± 851 | 377.5 ± 12 |
| 10 days | | | | | | |

(** P < 0.001, * P < 0.01, # P < 0.05 vs Baseline)
(& One rat (not included) developed severe uremia (BUN441, CRE10.8) @ 4 days and was dead at 10 days)
(# These rats received 20 mg/kg at 2 weeks after receiving 10 mg/kg)

### Example 6

### Studies of PQQ and Metoprolol's Effect on Cardiac Function

Pretreatment or treatment with 15 mg/kg of PQQ reduced infarct size and improved cardiac function in a rat model of ischemia/reperfusion (I/R). The beta-blocker metoprolol is used as standard treatment in patients with acute myocardial infarction. Accordingly, a experiments were conducted to study the combined treatment of myocardial infarction with metoprolol and low-dose PQQ compared to each drug alone. To determine mechanisms of cardioprotection, changes were measured in mitochondrial function and lipid peroxidation.

Intact male rats were subjected to 30 min of left anterior descending coronary artery occlusion and 2 hours of reperfusion with left ventricular hemodynamic monitoring. In preliminary experiments metoprolol at a dose of 1 mg/kg was found to be optimal in this system for infarct size reduction. Accordingly, metoprolol (1 mg/kg) and/or PQQ (3 mg/kg) was given by femoral vein injection at the onset of reperfusion to mimic clinical treatment. In separate experiments after ischemia/reperfusion, the mitochondrial respiratory control and ADP-to-oxygen consumption ratios (RCR) of the ischemic and non-ischemic myocardium were measured, as well as levels of malondialdehyde (MDA), an index of lipid peroxidation.

Results indicate that either treatment with metoprolol or PQQ reduced myocardial infarct size (infarct mass/risk area). The combined use of these agents tended to further reduce infarct size. Metoprolol and/or PQQ also protected against ischemia-induced left ventricular (LV) dysfunction after 1-2 hours of reperfusion. Thus, LV developed pressure was increased and LV end-diastolic pressure was decreased. Metoprolol and/or PQQ also reduced CK release. Mitochodrial RCR in ischemic and non-ischemic myocardium were enhanced primarily by PQQ, and less so by metoprolol. PQQ decreased MDA in ischemic and non-ischemic myocardium. These results are summarized in Table 2, below.

These experiments suggest that PQQ and metoprolol are effective in treating myocardial infarction, but the combination of PQQ and metoprolol may be more effective than either agent alone. For mitochondrial protection, PQQ is superior. It should be noted that in a recent large study of 45,852 patients with acute myocardial infarction randomized to metoprolol or placebo, the incidence of cardiogenic shock was about 30% higher in the metoprolol group (Collins R, et al. COMMIT/CCS-2; Placebo-controlled trial of early metoprolol in 46,000 acute myocardial infarction patients. Late-breaking trials presented at the American College of Cardiology Annual Scientific Session 2005. Mar. 6-9, 2005. Orlando, Fla.). This may be due at least in part to the inability of metoprolol to restore mitochondrial function.

TABLE 2

| | | | LV | | | | MDA |
|---|---|---|---|---|---|---|---|
| | | | Developed | | MT(RCR) | MDA | (nmol/g) |
| | Infarct size | LVEDP | Pressure | MT(RCR) | Non- | (nmol/g) | Non- |
| Groups | (%) | (mmHg) | (mmHg) | Ischemic | ischemic | Ischemic | ischemic |
| I/R | 39.9 ± 4.2 | 11.8 ± 2.1 | 78.3 ± 6.1 | 3.0 ± 0.5 | 5.7 ± 0.4 | 316 ± 88 | 237 ± 61 |
| (n = 9) | | | | | | | |
| MP | 26.5 ± 3.3** | 4.7 ± 3.0* | 87.7 ± 7.7 | 5.0 ± 0.7* | 7.2 ± 0.5* | | |
| (n = 6) | | | | | | | |
| PQQ | 24.3 ± 2.7** | 1.9 ± 1** | 89.1 ± 5.8 | 7.8 ± 0.3** | 8.0 ± 0.3** | 99 ± 14** | 118 ± 30* |
| (n = 12) | | | | | | | |
| MP + PQQ | 18.8 ± 1.1** | 3.0 ± 1.7** | 105 ± 2** | | | | |
| (n = 9) | | | | | | | |
| Sham | | | | 7.7 ± 0.3** | 7.9 ± .2** | 238 ± 26 | 206 ± 19 |
| (n = 7) | | | | | | | |
| Sham + PQQ | | | | 9.7 ± 0.2** | 10 ± 0.2** | 138 ± 15** | 128 ± 12* |
| (n = 4) | | | | | | | |

*P < 0.05,
**P < 0.01 vs Ischemia/Reperfusion (I/R)

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference.

1. A method of treating or preventing myocardial oxidative stress in a subject, comprising administering to a subject in need thereof a non-toxic amount of an agent which modulates myocardial oxidative stress such that one or more myocardial cells which are the subject of said oxidative stress are protected from cell death.

2. The method of claim 1, wherein said cell death is necrotic or apoptotic.

3. The method of claim 1, wherein said cell death is apoptotic.

4. The method of claim 1, wherein said cell death is necrotic.

5. The method of claim 1, wherein said myocardial oxidative stress is caused by hypoxia or ischemia.

6. The method of claim 1, wherein said agent is pyrroloquinoline quinone.

7. The method of claim 6, wherein said pyrroloquinoline quinone is administered at a dose between about 3 mg/kg and less than about 500 mg/kg.

8. The method of claim 6, wherein said pyrroloquinoline quinone is administered at a dose between about 15 mg/kg and about 100 mg/kg.

9. The method of claim 6, wherein said pyrroloquinoline quinone is administered at a dose of about 20 mg/kg.

10. A method of modulating a cardioprotective signaling pathway, comprising administering, to a patient in need of cardioprotection, pyrroloquinoline quinone in an amount effective to enhance or maintain the effect of said cardioprotective signaling pathway.

11. The method of claim 10, wherein said cardioprotective signaling pathway includes features an enzyme selected from the group consisting of RHO, PI 3-kinase, and Akt.

12. A method of modulating free radical damage caused by myocardial oxidative stress, comprising administering, to a patient in need of free radical myocardial damage, pyrroloquinoline quinone in a non-toxic amount effective to inhibit free radical damage.

13. The method of claim 12, wherein said free radical damage is inhibited by free radical scavenging mediated by PQQ.

14. The method of claim 12, wherein said free radical damage is inhibited by modulating free radical generation.

15. The method of claim 12, wherein said myocardial oxidative stress is caused by hypoxia or ischemia.

16. The method of claim 15, wherein said administration is done at the onset of reperfusion.

17. The method of claim 15, wherein said administration is done prior to reperfusion.

18. The method of claim 12, wherein said pyrroloquinoline quinone is administered at a dose between about 3 mg/kg and less than about 500 mg/kg.

19. The method of claim 12, wherein said pyrroloquinoline quinone is administered at a dose between about 15 mg/kg and about 100 mg/kg.

20. The method of claim 12, wherein said pyrroloquinoline quinone is administered at a dose of about 20 mg/kg.

21. A method of treating or preventing myocardial hypoxic or ischemic damage in a subject, comprising administering to a subject in need thereof a non-toxic amount of an agent which modulates myocardial hypoxic or ischemic damage such that myocardial cells are protected from cell death.

22. A method of improving coronary blood flow in a subject, comprising administering to a subject in need thereof a non-toxic amount of pyrroloquinoline quinone, such that coronary flow in said subject is improved.

23. The method of claim 22, wherein said coronary flow is determined by the LVDP.

24. The method of claim 22, wherein said coronary flow is determined by the LVEDP.

25. The method of claim 22, wherein said administration is done prior to reperfusion.

26. The method of claim 22, wherein said administration is done at the onset of reperfusion.

27. A method of treating or preventing cardiac injury caused by hypoxia or ischemia in a subject, comprising administering to a subject in need thereof pyrroloquinoline quinone, such that said hypoxia or ischemic-related injury is prevented or decreased, wherein said pyrroloquinoline quinone is administered at a dose of less than about 100 mg/kg.

28. The method of claim 27, wherein said injury is selected from the group consisting of myocardial infarction, cardiac arrest, and cardiac transplantation.

29. The method of claim 28, wherein said injury is myocardial infarction.

30. The method of claim 27, wherein said pyrroloquinoline quinone is administered at a dose between about 3 mg/kg and less than about 500 mg/kg.

31. The method of claim 27, wherein said pyrroloquinoline quinone is administered at a dose between about 15 mg/kg and about 100 mg/kg.

32. The method of claim 27, wherein said pyrroloquinoline quinone is administered at a dose of about 20 mg/kg.

33. The method of claim 27, wherein said injury is selected from the group consisting of myocardial infarction, cardiac arrest, and cardiac transplantation.

34. The method of claim 27, wherein said injury is myocardial infarction.

35. The method of claim 27, wherein said administration is done prior to reperfusion.

36. The method of claim 27, wherein said administration is done at the onset of reperfusion.

37. A method of treating or preventing cardiac injury caused by hypoxia or ischemia in a subject, comprising administering to a subject in need thereof pyrroloquinoline quinone, such that said hypoxia or ischemic-related injury is prevented or decreased, wherein said pyrroloquinoline quinone is administered at a non-toxic concentration.

38. The method of claim 37, wherein said pyrroloquinoline quinone is administered at a dose of about 20 mg/kg.

39. The method of claim 37, wherein said injury is selected from the group consisting of myocardial infarction; cardiac arrest; ischemia-reperfusion injury; congestive heart failure; cardiotoxicity; cardiac damage due to parasitic infection; fulminant cardiac amyloidosis; heart surgery; heart transplantation; and traumatic cardiac injury.

40. The method of claim 39, wherein said cardiotoxicity is caused by doxorubicin.

41. The method of claim 39, wherein said cardiac damage due to parasitic infection is via syphilis or chronic Trypanosoma cruzi infection.

42. The method of claim 39, wherein said traumatic cardiac injury is via penetrating or blunt cardiac injury, or aortic valve rupture.

43. A method of treating or preventing cardiac injury caused by hypoxia or ischemia in a subject, comprising administering to a subject in need thereof an NADPH-dependent methemoglobin reductase substrate, such that said cardiac injury is prevented or decreased.

44. The method of claim 43, wherein said NADPH-dependent methemoglobin reductase substrate is administered at a non-toxic concentration.

45. The method of claim 43, wherein said injury is selected from the group consisting of myocardial infarction; cardiac arrest; ischemia-reperfusion injury; congestive heart failure; cardiotoxicity; cardiac damage due to parasitic infection; fulminant cardiac amyloidosis; heart surgery; heart transplantation; and traumatic cardiac injury.

46. The method of claim 45, wherein said cardiotoxicity is caused by doxorubicin.

47. The method of claim 45, wherein said cardiac damage due to parasitic infection is via syphilis or chronic Trypanosoma cruzi infection.

48. The method of claim 45, wherein said traumatic cardiac injury is via penetrating or blunt cardiac injury, or aortic valve rupture.

49. A method of treating myocardial infarction, comprising administering to a subject in need thereof pyrroloquinoline quinone, such that said myocardial infarction is decreased or stabilized, wherein said pyrroloquinoline quinone is administered at a dose less than about 100 mg/kg.

50. A method of preventing organ damage during organ or tissue transplantation, comprising administering to an organ donor pyrroloquinoline quinone prior to or concurrent with removal of said organ, such that damage caused by reperfusion of said organ or tissue is decreased or prevented.

51. The method of claim 50, wherein said organ is the heart.

52. A method of preventing organ damage during organ or tissue transplantation, comprising contacting pyrroloquinoline quinone with one or more organs or tissues, such that damage caused by reperfusion of said organ or tissue is decreased or prevented.

53. A cardioprotective agent, comprising pyrroloquinoline quinone and a pharmaceutically acceptable carrier.

54. A kit for treating or preventing a hypoxia or ischemic-related cardiac injury, comprising in one or more containers pyrroloquinoline quinone, a pharmaceutically acceptable carrier, and instructions for use of said kit.

55. A method of preventing stroke in a subject suffering from heart failure, comprising treating said subject with pyrroloquinoline quinone and a pharmaceutically acceptable carrier, wherein said pyrroloquinoline quinone is administered at a dose of less than about 100 mg/kg.

56. A method of treating heart failure in a subject, comprising treating said subject with pyrroloquinoline quinone and a compound selected from the group consisting of anti-platelet drugs, anti-coagulant drugs, anti-thrombotic drugs, wherein said pyrroloquinoline quinone is administered at a dose of less than about 100 mg/kg.

57. A method of preventing vascular occlusion following balloon angioplasty in a subject, comprising pre-treating said subject with pyrroloquinoline quinone and a pharmaceutically acceptable carrier.

58. A method of reducing or preventing migraine headaches in a subject in need thereof, comprising treating said subject with pyrroloquinoline quinone and a pharmaceutically acceptable carrier, wherein said pyrroloquinoline quinone is administered at a dose of less than about 100 mg/kg.

59. A method of preventing reperfusion injury in a subject suffering from hypothermia, comprising pre-treating said subject with pyrroloquinoline quinone and a pharmaceutically acceptable carrier.

60. A pharmaceutical composition for treating myocardial infarction in a subject in need thereof, comprising a therapeutically effective dose of pyrroloquinoline quinone and a therapeutically effective dose of metoprolol.

61. The pharmaceutical composition of claim 60, wherein the therapeutically effective dose of pyrroloquinoline quinone is 3 mg/kg.

62. The pharmaceutical composition of claim 60, wherein the therapeutically effective dose of metoprolol is 1 mg/kg.

63. A kit for treating or preventing a hypoxia or ischemic-related cardiac injury, comprising in one or more containers pyrroloquinoline quinine, metoprolol, a pharmaceutically acceptable carrier, and instructions for use of said kit.

64. A method of treating or preventing myocardial oxidative stress in a subject, comprising administering to a subject in need thereof a therapeutically effective dose of pyrroloquinoline quinone and a therapeutically effective dose of metoprolol.

65. A method of treating or preventing myocardial infarction in a subject, comprising administering to a subject in need thereof a therapeutically effective dose of pyrroloquinoline quinone and a therapeutically effective dose of metoprolol.

66. A method of treating or preventing cardiac injury caused by hypoxia or ischemia in a subject, comprising administering to a subject in need thereof a therapeutically effective dose of pyrroloquinoline quinone and a therapeutically effective dose of metoprolol.
